SEQUENCE LISTING (16 sequences)

SEQ ID NO: 1 - DNA, 48 bp, artificial
  synthetic
tgaacactgaagctttctttggacaaggcaccagactcacagttgtag

SEQ ID NO: 2 - DNA, 48 bp, artificial
  synthetic
ctaactatggctacaccttcggttcggggaccaggttaaccgttgtag

SEQ ID NO: 3 - DNA, 50 bp, artificial
  synthetic
ctctggaaacaccatatattttggagagggaagttggctcactgttgtag

SEQ ID NO: 4 - DNA, 51 bp, artificial
  synthetic
caactaatgaaaaactgttttttggcagtggaacccagctctctgtcttgg

SEQ ID NO: 5 - DNA, 50 bp, artificial
  synthetic
tagcaatcagccccagcattttggtgatgggactcgactctccatcctag

SEQ ID NO: 6 - DNA, 53 bp, artificial
  synthetic
ctcctataattcacccctccactttgggaatgggaccaggctcactgtgacag

SEQ ID NO: 7 - DNA, 50 bp, artificial
  synthetic
ctcctacaatgagcagttcttcgggccagggacacggctcaccgtgctag

SEQ ID NO: 8 - DNA, 51 bp, artificial
  synthetic
cgaacaccggggagctgttttttggagaaggctctaggctgaccgtactgg

SEQ ID NO: 9 - DNA, 46 bp, artificial
  synthetic
ctgagaggcgctgctgggcgtctgggcggaggactcctggttctgg

SEQ ID NO: 10 - DNA, 49 bp, artificial
  synthetic
agcacagatacgcagtattttggcccaggcacccggctgacagtgctcg

SEQ ID NO: 11 - DNA, 50 bp, artificial
  synthetic
agccaaaaacattcagtacttcggcgccgggacccggctctcagtgctgg

SEQ ID NO: 12 - DNA, 48 bp, artificial
  synthetic
accaagagacccagtacttcgggccaggcacgcggctcctggtgctcg

SEQ ID NO: 13 - DNA, 53 bp, artificial
  synthetic
ctctggggccaacgtcctgactttcggggccggcagcaggctgaccgtgctgg

SEQ ID NO: 14 - DNA, 47 bp, artificial
  synthetic
ctcctacgagcagtacttcgggccgggcaccaggctcacggtcacag

SEQ ID NO: 15 - DNA, 387 bp, artificial
  synthetic
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagca

SEQ ID NO: 16 - DNA, 387 bp, artificial
  synthetic
gaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagca

(19) United States
(12) Patent Application Publication
(10) Pub. No.: US 2023/0295698 A1
(43) Pub. Date: Sep. 21, 2023

(54) COMPOSITIONS AND METHODS FOR DETERMINING T CELL CLONALITY

(71) Applicant: Advanced Cell Diagnostics, Inc., Newark, CA (US)

(72) Inventors: Xiao-Jun Ma, Pleasanton, CA (US); Wei Wei, Newark, CA (US); Brenda Nguyen, Milpitas, CA (US); Niyati Jhaveri, Palo Alto, CA (US); Alisa Baker, San Jose, CA (US); Robert Monroe, San Mateo, CA (US); Bingqing Zhang, Union City, CA (US)

(21) Appl. No.: 18/042,278

(22) PCT Filed: Aug. 19, 2021
(86) PCT No.: PCT/US21/46752

§ 371 Date: Feb. 20, 2023

Related U.S. Application Data
(60) Provisional application No. 63/068,611, filed on Aug. 21, 2020.

(51) Int. Cl. C12Q 1/6841

(52) U.S. Cl. CPC C12Q 1/6841

(57) ABSTRACT

The present disclosure provides compositions and methods related to the detection of mRNA variants. In particular, the present disclosure provides compositions and methods for detecting alternatively spliced mRNA variants using RNA in situ hybridization technology, which can be used to evaluate a disease state (e.g., malignancy) in a subject.

### CROSS REFERENCE TO RELATED APPLICATIONS

This application claims priority to and the benefit of U.S. Provisional Patent Application No. 63/068,611 filed Aug. 21, 2020, which is incorporated herein by reference in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 4.00 Kilobyte (3.957 Bytes) ASCII (Text) file named “Sequence_Listing_ST25.txt” created on Aug. 19, 2021.

### FIELD

The present disclosure provides compositions and methods related to the detection of mRNA variants. In particular, the present disclosure provides compositions and methods for detecting alternatively spliced mRNA variants using RNA in situ hybridization technology, which can be used to evaluate a disease state (e.g., malignancy) in a subject.

### BACKGROUND

Diseases such as cancer can initiate from a single malignant cell whose uncontrolled proliferation gives rise to a clonally related population of cancer cells. This hallmark property can be exploited as a powerful molecular marker for evaluating malignancy. For example, B cell and T cell clonality testing plays an important role in the diagnosis of non-Hodgkin lymphomas that are often difficult to differentiate from benign lymphoproliferative disorders. T cell lymphomas represent about 10-15% of all non-Hodgkin lymphomas in the North America, and accurately differentiating between benign lymphoproliferative disorders and T cell malignancies can often be difficult if based solely on morphological features and immunophenotypes. This has led to the development of T cell clonality testing methods to determine whether a suspected population of T cells are clonally related or not. Because T cells undergo an orderly process of genomic rearrangements in the T cell receptor (TCR) loci during their development, the tumor cells of a T cell malignancy generally all contain the same (i.e. clonal) genomic rearrangement events at the TCR loci. In contrast, a reactive T lymphocyte infiltrate will contain a heterogeneous population of T cells containing different (i.e. polyclonal) TCR rearrangements. TCR clonality analysis has evolved, from Southern blot analysis to PCR-based methods to next generation sequencing (NGS).

Currently, the standardized PCR protocols and PCR primers sets developed by the European BIOMED-2 Concerted Action BMH4-CT98-3936 are most widely used. The BIOMED-2 method uses multiple primer sets to amplify all possible junctional fragments arising from T cell receptor gene rearrangements, followed by analysis of the PCR products for heteroduplex formation or fragmentation patterns using gel and capillary electrophoresis to determine clonality. However, this method is technically complex, time-consuming and prone to misinterpretation.

### SUMMARY

Embodiments of the present disclosure include compositions and methods for detecting a target mRNA as a physiological indicator of a disease state or condition (e.g., T cell clonality). In accordance with these embodiments, the present disclosure provides a composition that includes a first target probe pool comprising a plurality of target probes, each target probe comprising a T section and an L section, wherein each T section is complementary to a portion of a first domain of a target mRNA and wherein each L section is complementary to a nucleic acid component of a signal generating complex. The composition also includes a first target probe comprising a T section and an L section, wherein the T section is complementary to a portion of a second domain of the target mRNA and wherein the L section is complementary to a nucleic acid component of a signal generating complex. The composition also includes a signal generating complex to generate a detectable signal corresponding to the target mRNA.

In some embodiments, each of the target probes of the first target probe pool includes a T section that is complementary to a non-overlapping portion of the first domain of the target mRNA. In some embodiments, the T section of the first target probe is complementary to a portion of a second domain of the target mRNA that is adjacent to the first domain of the target mRNA.

In some embodiments, the T section of at least one of the target probes of the first target probe pool is 3′ of its L section. In some embodiments, the T section of at least one of the target probes of the first target probe pool is 5′ of its L section. In some embodiments, the T section of the first target probe is 3′ of its L section. In some embodiments, the T section of the first target probe is 5′ of its L section. In some embodiments, at least one of the target probes of the first target probe pool and/or the first target probe form a hairpin structure.

In some embodiments, the signal generating complex includes a label probe, and optionally, one or more of an amplifier, a pre-amplifier, and a pre-pre-amplifier. In some embodiments, the label probe includes the nucleic acid component of the signal generating complex which binds the L section of at least one of the target probes of the first target probe pool and the L section of the first target probe. In some embodiments, the label probe includes at least one detectable label.

In some embodiments, the target mRNA includes a portion of a T cell receptor (TCR). In some embodiments, the first domain of the target mRNA includes Jβ1 or Jβ2 of a TCRβ 1 or a TCRβ2 mRNA, respectively. In some embodiments, the T sections of the plurality of target probes of the first target probe pool are complementary to at least a portion of any of SEQ ID NOs: 1-6. In some embodiments, the T sections of the plurality of target probes of the first target pool are complementary to at least a portion of any of SEQ ID NOs: 7-14.

In some embodiments, the second domain of the target mRNA includes a Cβ1 or a Cβ2 of a TCRβ1 or a TCRβ2 mRNA, respectively. In some embodiments, the T section of the first target probe is complementary to at least a portion of either of SEQ ID NOs: 15 or 16.

In some embodiments, the composition further includes a second target probe pool comprising a plurality of target probes, each target probe comprising a T section and an L section, wherein each T section is complementary to a portion of a first domain of a second target mRNA and wherein each L section is complementary to a nucleic acid component of a signal generating complex.

In some embodiments, the target mRNA complementary to the plurality of target probes in the first target probe pool is TCRβ1, and wherein the second target mRNA complementary to the plurality of target probes in the second target probe pool is TCRβ2.

In some embodiments, the composition further includes a second target probe comprising a T section and an L section, wherein the T section is complementary to a portion of a second domain of the second target mRNA, and wherein the L section is complementary to a nucleic acid component of a signal generating complex.

In some embodiments, the target mRNA complementary to the first target probe is TCRβ1, and wherein the second target mRNA complementary to the second target probe is TCRβ2.

In some embodiments, the composition further includes a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

Embodiments of the present disclosure also include a method of detecting an mRNA target. In accordance with these embodiments, the method includes (i) contacting a sample with a composition comprising a first target probe pool comprising a plurality of target probes, each target probe comprising a T section and an L section, wherein each T section is complementary to a portion of a first domain of a target mRNA and wherein each L section is complementary to a nucleic acid component of a signal generating complex; and a first target probe comprising a T section and an L section, wherein the T section is complementary to a portion of a second domain of the target mRNA and wherein the L section is complementary to a nucleic acid component of a signal generating complex; and a signal generating complex. The method also includes (ii) detecting a signal generated by the signal generating complex corresponding to the target mRNA in the sample.

In some embodiments, the method comprises RNA in situ hybridization. In some embodiments, the method comprises hybridization chain reaction.

In some embodiments, one of the plurality of target probes of the first target probe pool binds the target mRNA.

In some embodiments, the nucleic acid portion of the signal generating complex binds both the L section of one of the plurality of target probes of the first target probe pool and the L section of the first target probe.

In some embodiments, the composition further includes a second target probe pool comprising a plurality of target probes, each target probe comprising a T section and an L section, wherein each T section is complementary to a portion of a first domain of a second target mRNA and wherein each L section is complementary to a nucleic acid component of a signal generating complex; and a second target probe comprising a T section and an L section, wherein the T section is complementary to a portion of a second domain of the second target mRNA, and wherein the L section is complementary to a nucleic acid component of a signal generating complex.

In some embodiments, first domain of the target mRNA comprises Jβ1 of a TCRβ1 mRNA and the second domain of the target mRNA comprises C131 of a TCRβ 1 mRNA; and wherein the first domain of the second target mRNA comprises Jβ2 of a TCRβ2 mRNA and the second domain of the second target mRNA comprises Cβ2 of a TCRβ2 mRNA.

In some embodiments, the method further includes detecting the TCRβ1 mRNA and the TCRβ2 mRNA in the sample simultaneously using different labels. In some embodiments, the method includes detecting the TCRβ1 mRNA and the TCRβ2 mRNA in the sample separately using the same or different labels.

In some embodiments, the method further includes evaluating clonality of cells in the sample based on the proportion of TCRβ1 mRNA and TCRβ2 mRNA detected in the sample.

Embodiments of the present disclosure also include a kit for detecting an mRNA target. In accordance with these embodiments, the kit includes a first target probe pool comprising a plurality of target probes, each target probe comprising a T section and an L section, wherein each T section is complementary to a portion of a first domain of a target mRNA and wherein each L section is complementary to a nucleic acid component of a signal generating complex; a first target probe comprising a T section and an L section, wherein the T section is complementary to a portion of a second domain of the target mRNA and wherein the L section is complementary to a nucleic acid component of a signal generating complex; a signal generating complex; and instructions for performing a hybridization reaction to detect the mRNA target.

In some embodiments, the signal generating complex includes a label probe, and optionally, one or more of an amplifier, a pre-amplifier, and a pre-pre-amplifier. In some embodiments, the label probe includes at least one detectable label.

In some embodiments, the kit further includes at least one of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

In some embodiments, the kit further includes a calibrator or control polynucleotide. In some embodiments, the calibrator or control polynucleotide comprises a sequence complementary to a portion of any one of SEQ ID NOs: 1-16. In some embodiments, the calibrator or control polynucleotide comprises a sequence identical to a portion of any one of SEQ ID NOs: 1-16.

In some embodiments, the kit further includes a second target probe pool comprising a plurality of target probes, each target probe comprising a T section and an L section, wherein each T section is complementary to a portion of a first domain of a second target mRNA and wherein each L section is complementary to a nucleic acid component of a signal generating complex; and a second target probe comprising a T section and an L section, wherein the T section is complementary to a portion of a second domain of the second target mRNA, and wherein the L section is complementary to a nucleic acid component of a signal generating complex.

Embodiments of the present disclosure also include a method for performing a T cell clonality assay. In accordance with these embodiments, the method includes (i) contacting a sample with a composition comprising: a first target probe pool comprising a plurality of target probes, each target probe comprising a T section and an L section, wherein each T section is complementary to a portion of a first domain of a target mRNA and wherein each L section is complementary to a nucleic acid component of a signal generating complex; a first target probe comprising a T section and an L section, wherein the T section is complementary to a portion of a second domain of the target mRNA and wherein the L section is complementary to a nucleic acid component of a signal generating complex; a second target probe pool comprising a plurality of target probes, each target probe comprising a T section and an L section, wherein each T section is complementary to a portion of a first domain of a second target mRNA and wherein each L section is complementary to a nucleic acid component of a signal generating complex; a second target probe comprising a T section and an L section, wherein the T section is complementary to a portion of a second domain of the second target mRNA, and wherein the L section is complementary to a nucleic acid component of a signal generating complex; and a signal generating complex; and (ii) detecting a signal generated by the signal generating complex corresponding to the target mRNA in the sample, and detecting a different signal generated by the signal generating complex corresponding to the second target mRNA in the sample. In some embodiments, T cell clonality is determined based on the proportion of each signal generated.

In some embodiments, the assay comprises RNA in situ hybridization. In some embodiments, the assay comprises hybridization chain reaction.

In some embodiments, one of the plurality of target probes of the first target probe pool binds the target mRNA, and one of the plurality of target probes of the second target probe pool binds the second target mRNA.

In some embodiments, the first domain of the target mRNA comprises Jβ1 of a TCRβ1 mRNA and the second domain of the target mRNA comprises Cβ1 of a TCRβ1 mRNA; and wherein the first domain of the second target mRNA comprises Jβ2 of a TCRβ2 mRNA and the second domain of the second target mRNA comprises Cβ2 of a TCRβ2 mRNA.

In some embodiments, the sample comprises at least one of cell lysate, cell culture, a cell line, a tissue sample, an organ, an organelle, a biological fluid, a mucosa sample, a blood sample, a plasma sample, a urine sample, a skin tissue sample, a vascular tissue sample, a pancreatic tissue sample, a lymphoid tissue sample, a tumor tissue sample, T cell lymphoma tissue, T cells, B cells, and any combination thereof.

In some embodiments, determining T cell clonality based on the proportion of each signal generated includes quantifying the signals and comparing relative levels of signals of TCRβ 1 and TCRβ2 mRNAs. In some embodiments, a predominance of either one of these two signals in a population of cells indicates T cell mono-clonality or clonality, whereas lack of dominance of either signal in a population of cells indicate T cell polyclonality or non-clonality.

In some embodiments, the sample is a fixed tissue sample, and wherein determining T cell clonality based on the proportion of each signal generated comprises assessing spatial distribution of the signals within the tissue sample. The predominance of either TCRβ1 or TCRβ2 signal in a region of interest indicates T cell mono-clonality or clonality, whereas a lack of dominance of either signal in the region of interest indicates T cell poly-clonality or non-clonality.

In some embodiments, the method further includes administering a treatment based on determining T cell clonality.

In some embodiments, administering a treatment includes CAR T-Cell therapy, chemotherapy, immunotherapy, radiation, drug treatment, stem cell transplantation, surgery, and any combination thereof.

Embodiments of the present disclosure also include a composition for use in diagnosing lymphoma. In accordance with these embodiments, the composition includes a first target probe pool comprising a plurality of target probes, each target probe comprising a T section and an L section, wherein each T section is complementary to a portion of a first domain of a target mRNA and wherein each L section is complementary to a nucleic acid component of a signal generating complex; a first target probe comprising a T section and an L section, wherein the T section is complementary to a portion of a second domain of the target mRNA and wherein the L section is complementary to a nucleic acid component of a signal generating complex; a second target probe pool comprising a plurality of target probes, each target probe comprising a T section and an L section, wherein each T section is complementary to a portion of a first domain of a second target mRNA and wherein each L section is complementary to a nucleic acid component of a signal generating complex; a second target probe comprising a T section and an L section, wherein the T section is complementary to a portion of a second domain of the second target mRNA, and wherein the L section is complementary to a nucleic acid component of a signal generating complex; and a signal generating complex.

In some embodiments, a first signal is generated corresponding to an amount of the target mRNA in the sample and wherein a second signal is generated corresponding to an amount of the second target mRNA in the sample, and wherein the subject is diagnosed as having a lymphoma based on a comparison of the signals generated.

Other aspects and embodiments of the disclosure will be apparent in light of the following detailed description.

### BRIEF DESCRIPTION OF THE DRAWINGS

FIGS. 1A-1B: Pathway of T cell receptor β locus rearrangements and RNA splicing produces mature TCRB mRNA, TCRB1. (FIG. 1A). The first rearrangements of the T cell receptor β locus may be nonproductive due to out-of-frame joining of D-J or V-DJ segments. The rescue pathway uses the D-J-C β2 elements to produce an alternative TCRB mRNA, TCRB2 (FIG. 1B).

FIG. 2: Representative probe design strategy to differentiate between TCRB1 and TCRB2 mRNAs using Jβ1 or Jβ2 target probe pools in conjunction with probes targeting either Cβ1 or Cβ2 elements.

FIG. 3: Detection of TCRB1 and TCRB2 mRNA with a duplex assay. TCRB1 mRNA was detected with a green chromogen using a 0.1131 probe pool and a Cβ 1 oligo, as shown in FIG. 2, and TCRB2 mRNA was detected with a red chromogen using a Jβ2 probe pool and a Cβ2 oligo (FIG. 2). The two T cell lymphoma-derived cell lines Jurkat and CCRF demonstrated virtually pure green and pure red chromogen signals, respectively, consistent with their monoclonal nature. In contrast, the T cells in the human tonsil tissue demonstrated a heterogeneous population of cells that stained either green or red in a mutually exclusive manner, consistent with the expected polyclonal populations of T cells in the tonsil.

FIG. 4: Detection of TCRB clonality in a T cell lymphoma tissue with the duplex assay. The Jβ1 probe pool and a Cβ1 oligo, as shown in FIG. 2, were used to detect TCRB1 mRNA with a green chromogen, and the Jβ2 probe pool and a Cβ2 oligo (FIG. 2) were used to detect TCRB2 mRNA with a red chromogen. The assay detected virtually exclusively green chromogen signals in this tumor tissue, indicating a clonal T cell origin involving Jβ1 rearrangements (TCRB1).

### DETAILED DESCRIPTION

The present disclosure provides compositions and methods related to the detection of mRNA variants. In particular, the present disclosure provides compositions and methods for detecting alternatively spliced mRNA variants using RNA in situ hybridization (RISH) technology, which can be used to evaluate a disease state (e.g., malignancy) in a subject.

In accordance with these embodiments, the compositions and methods disclosed herein exploit the sequence differences among mRNA transcript variants that are differentially present in malignant and non-malignant cells. In one embodiment, the compositions and methods provided herein detect the Jβ1 and the Jβ2 segments in the T cell receptor β (TCRB) locus to detect the alternative usage of Jβ1 and Jβ2 segments in fully spliced TCRB mRNA in a population of T cells. In one embodiment, nucleic acid probes specific for each of the Jβ1 and the Jβ2 segments are combined with a nucleic acid probe targeting either Cβ1 or Cβ2 in a paired probe strategy.

This strategy enables robust signal amplification while maintaining high specificity due to the requirement for the simultaneous hybridization of two oligo probes in tandem to the target RNA. This RISH-based method avoids the common limitations associated with the current PCR-based methods such as: (i) PCR amplification bias due to different amplicon sizes which may exhibit differential sensitivity to DNA fragmentation (especially in FFPE tissue); (ii) false-positive results of pseudo-clonality due to limited DNA input; and (iii) misinterpretation of fragment pattern data. The RISH-based methods and compositions disclosed herein also provide the following benefits: (i) provide a simpler assay that can be fully automated in a clinical lab setting; (ii) accurate results that are straightforward to interpret; (iii) applicable to highly heterogeneous samples (e.g., scarce tumor content in a sample); and (iv) rapid turnaround time (e.g., within a single day).

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the present disclosure may be readily combined, without departing from the scope or spirit of the embodiments provided herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “probe” as used herein refers to a capture agent that is directed to a specific target mRNA sequence. Accordingly, each probe of a probe set has a respective target mRNA sequence. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A “single-stranded nucleic acid” having secondary structure (e.g., base-paired secondary structure) and/or higher order structure comprises a “double-stranded nucleic acid.” For example, triplex structures are considered to be “double-stranded.” In some embodiments, any base-paired nucleic acid is a “double-stranded nucleic acid.”

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. RNA can be spliced in various ways to produce “alternatively spliced mRNA” transcripts that can encode different polypeptides with different functions. In some aspects, pre-mRNA may be differentially spliced during processing of the pre-mRNA to produce various forms of alternatively spliced RNA, any of which may produce a mature mRNA transcript that encodes a polypeptide. As would be recognized by one of ordinary skill in the art based on the present disclosure, the methods and compositions provided herein can be used to detect any species of alternatively spliced pre-mRNA or mature mRNA transcripts, and differentiate it from other species of alternatively spliced pre-mRNA or mature mRNA transcripts. Additionally, the methods and compositions provided herein can be used to detect any species of alternative pre-mRNA or mature mRNA transcripts that are transcribed from alternatively recombined DNA sequences through DNA recombination mechanisms (e.g., at the TCR loci) or genetic engineering or disease mechanisms, and differentiate these species of alternative pre-mRNA or mature mRNA transcripts from other species of alternative pre-mRNA or mature mRNA transcripts. Additionally, one of skill in the art would recognize that the methods and compositions described herein can be used to detect any target nucleic acid molecule, including different forms of DNA target molecules such as those having DNA rearrangements (e.g., chromosomal rearrangements, e.g., which can give rise to a disease/condition (e.g., muscular dystrophy)), The methods and compositions provided herein can therefore be used to differentiate any nucleic acid target molecule from non-target species based on alternative nucleic acid sequences.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

In some embodiments the substitutions can be conservative amino acid substitutions. Examples of conservative amino acid substitutions, unlikely to affect biological activity, include the following: alanine for serine, valine for isoleucine, aspartate for glutamate, threonine for serine, alanine for glycine, alanine for threonine, serine for asparagine, alanine for valine, serine for glycine, tyrosine for phenylalanine, alanine for proline, lysine for arginine, aspartate for asparagine, leucine for isoleucine, leucine for valine, alanine for glutamate, aspartate for glycine, and these changes in the reverse. See e.g., Neurath et al., The Proteins, Academic Press, New York (1979), the relevant portions of which are incorporated herein by reference. Further, an exchange of one amino acid within a group for another amino acid within the same group is a conservative substitution, where the groups are the following: (1) alanine, valine, leucine, isoleucine, methionine, norleucine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3‘-T-C-A-S’.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary,” “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, and cells isolated from a mammal Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, and the like.

As used herein, and unless otherwise specified, the terms “treat,” “treating,” and “treatment” refer to an action that occurs while a patient is suffering from the specified cancer, which reduces the severity of the cancer or retards or slows the progression of the cancer.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician. An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The terms “detecting” as used herein generally refer to any form of measurement, and include determining whether an element is present or not. This term includes quantitative and/or qualitative determinations.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Compositions

Embodiments of the present disclosure provide compositions and methods related to detecting one or more target mRNAs in a sample. In some embodiments, of the compositions provided herein enable the detection of a target mRNA using in situ hybridization (ISH), including detecting any variant species of a given mRNA target, including pre-mRNA and mature mRNA targets. The RNA in situ hybridization approaches described herein address the deficiencies of many currently available approaches for detecting RNA and assessing gene expression patterns or cellular phenotypes generally. For example, one advantage of the embodiments of the compositions of the present disclosure is that they can be performed using lab automation common in anatomic pathology laboratories, making them widely accessible and practical for testing larger numbers of cell and tissue samples. Other techniques such as next-generation sequencing (NGS), PCR, and DNA fluorescent in situ hybridization (FISH) are not as widely available and/or involve well-documented technical limitations.

In accordance with these embodiments, the present disclosure includes compositions and methods for detecting a target mRNA as a physiological indicator of a disease state or condition (e.g., T cell clonality). In some embodiments, the present disclosure provides a composition that includes a first target probe pool comprising a plurality of target probes. As used herein, a probe comprising a nucleic acid sequence complementary to a target region generally refers to a probe having a sequence that is at least partially complementary to the target region. Thus, the probe may contain additional nucleic acids that are not complementary to that region.

In accordance with these embodiments, the present disclosure includes compositions for detecting an mRNA target, and/or distinguishing the mRNA target from one or more variants (e.g., splice variants). In some embodiments, the composition includes a first target probe pool comprising a plurality of target probes. Each target probe in the target probe pool comprising a target (T) section and a label (L) section. Each T section is complementary to a portion of a first domain of a target mRNA and each L section is complementary to a nucleic acid component of a signal generating complex (SGC). In some embodiments, the T sections of the two or more target probes in a target probe pool are complementary to non-overlapping regions of the target nucleic acid, and the L sections of the two or more target probes are complementary to non-overlapping regions of the nucleic acid component of the generating complex.

In some embodiments, one of the plurality of target probes of a target probe pool binds the target mRNA. For example, only one of the plurality of target probes of a target probe pool binds a portion of a domain in a target mRNA, whereas the other target probes in the target probe pool do not bind the target mRNA (e.g., the portion of the domain of the target mRNA is not present in the mature target mRNA being detected). The presence of multiple target probes in the target probe pool facilitates the potential detection of any splice variants of the target mRNA, and in some embodiments, can be used as part of a method of detecting a disease state or condition (described further below).

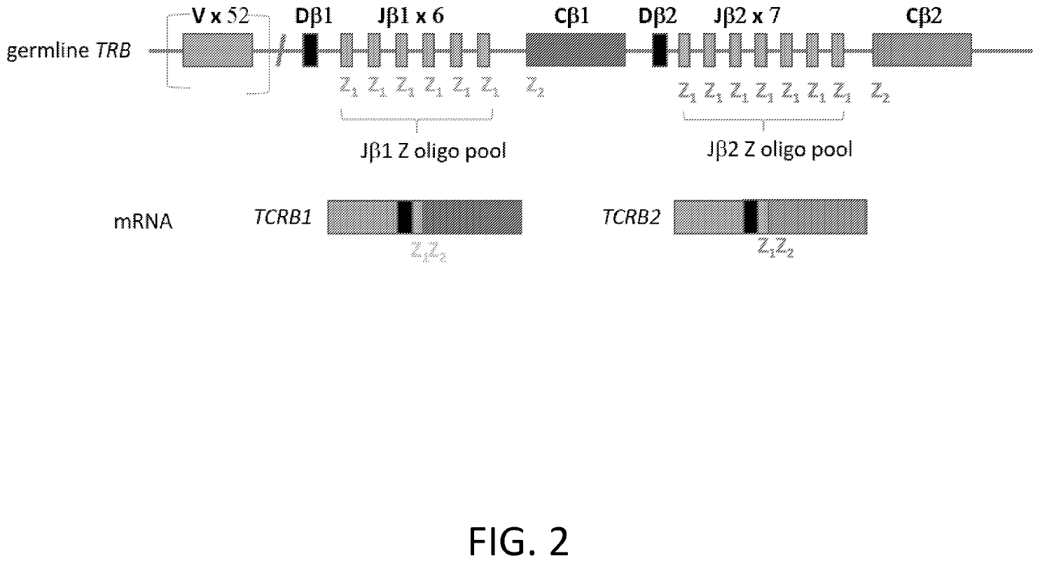

In some embodiments, the composition for detecting a target mRNA also includes a first target probe comprising a T section and an L section. The T section is complementary to a portion of a second domain of the target mRNA and the L section is complementary to a nucleic acid component of a signal generating complex. For example, the T section of a target probe can be complementary to a portion of a domain of a target mRNA, such that this domain is adjacent to the domain to which a target probe of the target probe pool binds (FIG. 2). Thus, a given target mRNA can be detected using a single pair of probes (e.g., one target probe from a target probe pool binding a portion of a first domain and a target probe binding a portion of a second domain that is adjacent to the first domain).

In some embodiments, the first domain and the second domain represent separate exons that are present in a pre-mRNA, but may be differentially spiced during processing of the pre-mRNA into a mature mRNA that can be the target mRNA. In some embodiments, the target probes each bind separate portions of distinct exons, flanking an intron-exon boundary. For example, the target probes in the target probe pool can each be designed to bind a portion of Jβ1 or Jβ2 of a TCRβ1 or a TCRβ2 mRNA, respectively (e.g., SEQ ID NOs: 1-14), and the corresponding target probe can be designed to bind a portion of Cβ1 or Cβ2 of a TCRβ1 or a TCRβ2 mRNA, respectively (SEQ ID NOs: 15 and 16). Alternatively, the target probes in the target probe pool can each be designed to bind a portion of Jβ1 or Jβ2 of a TCRβ1 or a TCRβ2 mRNA, respectively (e.g., SEQ ID NOs: 1-14), and the corresponding target probe can be designed to bind a portion of Dβ1 or Dβ2 of a TCRβ1 or a TCRβ2 mRNA, respectively.

The hybridization or binding of the probes to their respective T sections in a target mRNA facilitates the generation of two adjacent L sections to which a complementary nucleic acid component of a signal generating complex (SGC) can bind. In some embodiments, the nucleic acid portion of the signal generating complex binds both the L section of one of the plurality of target probes of the first target probe pool and the L section of the first target probe. In some embodiments, a nucleic acid portion of the signal generating complex binds the L section of one of the plurality of target probes of the first target probe pool and a separate nucleic acid component binds the L section of the first target probe.

In some embodiments, once the SGC binds the target probes at their L sections, other components of the SGC can bind. In some embodiments, the SGC provided herein comprises additional components, including but not limited to an amplifier, a pre-amplifier, and/or a pre-pre-amplifier. In accordance with these embodiments, the formation of the SGC allows for the generation of a detectable signal that is indicative of the presence of the target mRNA. Detecting the mRNA using a SGC and/or a label probe an occur by means well known in the art, including but not limited to, chromogenic or fluorescent detection means (described further below).

In some embodiments, the T section of at least one of the target probes of the first target probe pool is 3′ of its L section. In some embodiments, the T section of at least one of the target probes of the first target probe pool is 5′ of its L section. In some embodiments, the T section of the first target probe is 3′ of its L section. In some embodiments, the T section of the first target probe is 5′ of its L section. In some embodiments, the T sections of all of the target probes of the first target probe pool are 3′ of their L sections. In some embodiments, the T sections of all of the target probes of the first target probe pool are 5′ of their L sections. In some embodiments, the T section of at least one of the target probes of the first target probe pool is 3′ of its L section, and the T section of the first target probe is 3′ of its L section, or vice versa (e.g., both probes have the same directionality). In some embodiments, the T section of at least one of the target probes of the first target probe pool is 5′ of its L section, and the T section of the first target probe is 5′ of its L section, or vice versa (e.g., both probes have the same directionality). In some embodiments, the T section of at least one of the target probes of the first target probe pool is 5′ of its L section, and the T section of the first target probe is 3′ of its L section, or vice versa (e.g., one probe has directionality that is opposite the other). In some embodiments, the T section of at least one of the target probes of the first target probe pool is 3′ of its L section, and the T section of the first target probe is 5′ of its L section, or vice versa (e.g., one probe has directionality that is opposite the other).

In some embodiments, at least one of the target probes of the first target probe pool and/or the first target probe form a hairpin structure (e.g., useful for performing an assay involving hybridization chain reaction). In some embodiments, the plurality of target probes of in a target probe pool and/or the separate target probe each comprises a non-binding portion separating its T section from its L section (e.g., a spacer or linker region). In some embodiments, this non-binding portion does not bind to either the target mRNA or the nucleic acid component of the SGC, but is useful in forming the SGC. In some embodiments, this non binding region can form the unpaired loop section of a hairpin loop. In other embodiments, neither the target probes in the target probe pool nor the separate target probe contains a non binding portion.

In some embodiments, the SGC includes a label probe, and optionally, one or more of an amplifier, a pre-amplifier, and a pre-pre-amplifier. In some embodiments, the label probe includes the nucleic acid component of the SGC, which binds the L section of at least one of the target probes of the first target probe pool and the L section of the first target probe. In some embodiments, the label probe includes at least one detectable label (e.g., chromogenic or fluorescent detection means).

In some embodiments, the target mRNA includes a portion of a T cell receptor (TCR). In some embodiments, the first domain of the target mRNA includes Jβ1 or Jβ2 of a TCRβ 1 or a TCRβ2 mRNA, respectively. In some embodiments, the T sections of the plurality of target probes of the first target probe pool are complementary to at least a portion of any of SEQ ID NOs: 1-6. In some embodiments, the T sections of the plurality of target probes of the first target pool are complementary to at least a portion of any of SEQ ID NOs: 7-14.

In some embodiments, the second domain of the target mRNA includes a Cβ1 or a Cβ2 of a TCRβ1 or a TCRβ2 mRNA, respectively. In some embodiments, the T section of the first target probe is complementary to at least a portion of either of SEQ ID NOs: 15 or 16.

In some embodiments, the composition further includes a second target probe pool comprising a plurality of target probes. Each of the target probes includes a T section and an L section, each T section being complementary to a portion of a first domain of a second target mRNA and each L section being complementary to a nucleic acid component of an SGC. In some embodiments, the target mRNA complementary to the plurality of target probes in the first target probe pool is TCRβ1, and the second target mRNA complementary to the plurality of target probes in the second target probe pool is TCRβ2 (e.g., for performing a T cell clonality assay).

In some embodiments, the composition further includes a second target probe comprising a T section and an L section. The T section is complementary to a portion of a second domain of the second target mRNA, and the L section is complementary to a nucleic acid component of an SGC. In some embodiments, the target mRNA complementary to the first target probe is TCRβ1, and the second target mRNA complementary to the second target probe is TCRβ2.

In some embodiments, the composition further includes one or more components useful for carrying out a nucleic acid hybridization reaction, such as an in situ hybridization reaction or a hybridization chain reaction assay. The composition can include, but is not limited to, one or more of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

In accordance with these embodiments, the “target probes” contained in the compositions of the present disclosure generally refer to a polynucleotide that is capable of hybridizing to a target nucleic acid (e.g., pre-mRNA and/or mature mRNA) and capturing or binding a label probe or SGC component to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid (T section) and a second polynucleotide sequence that is complementary to a polynucleotide sequence of the label probe (L section), amplifier, pre-amplifier, pre-pre amplifier, or the like. The target probe is generally single-stranded so that the complementary sequence is available to hybridize with a corresponding target nucleic acid, label probe, amplifier, pre-amplifier or pre-pre-amplifier. In some embodiments, the target probes are provided as a pair. For example, a target probe pair can be designed to bind separate domains of a mRNA target (e.g., flanking a splice junction), with each probe having forward or reverse directionality independent of the other target probe in the pair.

In some embodiments, the compositions of the present disclosure include a “label probe,” which generally refers to a polynucleotide that binds to a target molecule, directly or indirectly, generally indirectly, and allows the target to be detected. A label probe contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. In some embodiments, the label probe can comprise at least one polynucleotide sequence that is complementary to a polynucleotide sequence in an amplifier, pre-amplifier, or pre-pre-amplifier in an SGC. In some embodiments, the SGC provided herein comprises additional components such an amplifier, a pre-amplifier, and/or a pre-pre-amplifier.

In some embodiments, the compositions of the present disclosure include an “amplifier,” which is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides described herein, the amplifier can include modified nucleotides and/or nonstandard inter-nucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated herein by reference in their entirety.

In some embodiments, the compositions of the present disclosure include a “pre-amplifier,” which is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated herein by reference in its entirety.

In some embodiments, the compositions of the present disclosure include a “pre-pre-amplifier,” which is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated herein by reference in its entirety.

### 3. Methods of Detection

Embodiments of the methods provided herein include methods of detecting a target mRNA using in situ hybridization (ISH). These detection methods can include detecting any variant species of a given mRNA target, including pre-mRNA and mature mRNA. The RNA in situ hybridization approaches described herein address the deficiencies of many currently available approaches for detecting RNA and assessing gene expression patterns or cellular phenotypes generally. For example, one advantage of the embodiments of the present disclosure is that they can be performed using lab automation common in anatomic pathology laboratories, making them widely accessible and practical for testing larger numbers of cell and tissue samples. Other techniques such as next-generation sequencing (NGS), PCR, and DNA fluorescent in situ hybridization (FISH) are not as widely available and/or involve well-documented technical limitations. In addition, the present methods are much more cost-effective and efficient.

Additionally, the methods provided herein produce data that are amenable to rapid and accurate interpretation, such as chromogenic dot signals indicating of positive gene expression for a given mRNA target. In many instances, the signals generated using these methods are easier to interpret and quantify than immunohistochemistry (IHC) staining, as they are capable of providing single-molecule resolution. Unlike the sometimes weak signals from IHC, the chromogenic dots resulting from detection of a target RNA in the context of the methods provided herein are easily identified and quantified, without any significant background interference, which can interfere with data interpretation.

The methods provided herein generally relate to RNA in situ detection of target nucleic acids (e.g., TCRB mRNA). Methods for in situ detection of nucleic acids are well known to those skilled in the art (see, for example, US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, such as mRNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides.

In one embodiment, the RNA ISH (RISH) used herein to detect mRNA targets is RNAscope®, which is described in more detail in, e.g., U.S. Pat. Nos. 7,709,198, 8,604,182, and 8,951,726, which are herein incorporated by reference in their entireties. Specifically, RNAscope® describes using specially designed oligonucleotide probes, sometimes referred to as “double-Z” or ZZ probes, in combination with a branched-DNA-like signal amplification system to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012)). Such a probe design greatly improves the specificity of signal amplification because only when both probes in each pair bind to their intended target can signal amplification occur.

In one embodiment, the RNA ISH (RISH) used herein to detect mRNA targets is BaseScope™, which is described in more detail in, e.g., U.S. patent application Ser. No. 13/575,936, and PCT Appln. No. PCT/US2011/023126, which are incorporated herein by reference in their entireties. Specifically, BaseScope™ includes the use of specially designed oligonucleotide probes, sometimes referred to as “double-Z” or ZZ probes, in combination with a branched-DNA-like signal amplification system to reliably detect target RNA with single-molecule sensitivity under standard bright-field microscopy. The BaseScope™ platform enables applications such as the detection of exon junctions/splice variants, short/highly homologous RNA sequences (50-300 bases), and point mutations at single cell sensitivity (Anderson, C. M. et al. Visualizing Genetic Variants, Short Targets, and Point Mutations in the Morphological Tissue Context with an RNA In Situ Hybridization Assay. J. Vis. Exp. (2018); doi:10.3791/58097). Such a probe design greatly improves the specificity of signal amplification because only when both probes in each pair bind to their intended target can signal amplification occur, enabling the detecting of biological events in cells and in situ using a single Z probe pair.

In another embodiment, the RNA ISH methods of the present disclosure include the use of probes that form stable DNA hairpins, along with a DNA initiator probe. These probes can be used to detect a target mRNA using a hybridization chain reaction (HCR) mechanism. The addition of an initiator strand of DNA to the stable mixture of two hairpin species triggers a chain reaction of hybridization events between the hairpins, which is used to amplify a detectable signal (see, e.g., Dirks, R. M. and Pierce, N. A. Triggered amplification by hybridization chain reaction. Proc. Natl. Acad. Sci. USA 101, 15275-15278 (2004)).

In accordance with these embodiments, the present disclosure includes a method of detecting an mRNA target, and/or distinguishing the mRNA target from one or more variants (e.g., splice variants). In some embodiments, the method includes contacting a sample with a composition comprising a first target probe pool comprising a plurality of target probes. As described further above, each target probe in the target probe pool comprising a target (T) section and a label (L) section. Each T section is complementary to a portion of a first domain of a target mRNA and each L section is complementary to a nucleic acid component of a signal generating complex.

In some embodiments, the T sections of the two or more target probes in a target probe pool are complementary to non-overlapping regions of the target nucleic acid, and the L sections of the two or more target probes are complementary to non-overlapping regions of the nucleic acid component of the generating complex. In some embodiments, one of the plurality of target probes of a target probe pool binds the target mRNA. For example, only one of the plurality of target probes of a target probe pool binds a portion of a domain in a target mRNA, whereas the other target probes in the target probe pool do not bind the target mRNA (e.g., the portion of the domain of the target mRNA is not present in the mature target mRNA being detected). The presence of multiple target probes in the target probe pool facilitates the potential detection of any splice variants of the target mRNA, and in some embodiments, can be used as part of a method of detecting a disease state or condition.

As described further herein, the composition for detecting a target mRNA includes a first target probe comprising a T section and an L section. The T section is complementary to a portion of a second domain of the target mRNA and the L section is complementary to a nucleic acid component of a signal generating complex. For example, the T section of a target probe can be complementary to a portion of a domain of a target mRNA, such that this domain is adjacent to the domain to which a target probe of the target probe pool binds (FIG. 2). Thus, a given target mRNA can be detected using a single pair of probes (e.g., one target probe from a target probe pool binding a portion of a first domain and a target probe binding a portion of a second domain that is adjacent to the first domain).

In some embodiments, the first domain and the second domain represent separate exons that are present in a pre-mRNA, but may be differentially spiced during processing of the pre-mRNA into a mature mRNA that can be the target mRNA. In some embodiments, the target probes each bind separate portions of distinct exons, flanking an intron-exon boundary. For example, the target probes in the target probe pool can each be designed to bind a portion of Jβ1 or Jβ2 of a TCRβ1 or a TCRβ2 mRNA, respectively (e.g., SEQ ID NOs: 1-14), and the corresponding target probe can be designed to bind a portion of Cβ1 or Cβ2 of a TCRβ1 or a TCRβ2 mRNA, respectively (SEQ ID NOs: 15 and 16). Alternatively, the target probes in the target probe pool can each be designed to bind a portion of Jβ1 or Jβ2 of a TCRβ1 or a TCRβ2 mRNA, respectively (e.g., SEQ ID NOs: 1-14), and the corresponding target probe can be designed to bind a portion of Dβ1 or Dβ2 of a TCRβ1 or a TCRβ2 mRNA, respectively.

The hybridization or binding of the probes to their respective T sections in a target mRNA facilitates the generation of two adjacent L sections to which a complementary nucleic acid component of a signal generating complex (SGC) can bind. In some embodiments, the nucleic acid portion of the signal generating complex binds both the L section of one of the plurality of target probes of the first target probe pool and the L section of the first target probe. In some embodiments, a nucleic acid portion of the signal generating complex binds the L section of one of the plurality of target probes of the first target probe pool and a separate nucleic acid component binds the L section of the first target probe.

In some embodiments, once the SGC binds the target probes at their L sections, other components of the SGC can bind. In some embodiments, the SGC provided herein comprises additional components, including but not limited to an amplifier, a pre-amplifier, and/or a pre-pre-amplifier. In accordance with these embodiments, the formation of the SGC allows for the generation of a detectable signal that is indicative of the presence of the target mRNA. Detecting the mRNA using a SGC and/or a label probe an occur by means well known in the art, including but not limited to, chromogenic or fluorescent detection means (described further below).

In some embodiments, the methods of detecting mRNA target(s) provided herein include use of a composition comprising a second target probe pool comprising a plurality of target probes, each target probe comprising a T section and an L section. Each T section is complementary to a portion of a first domain of a second target mRNA and each L section is complementary to a nucleic acid component of a signal generating complex. The composition also includes a second target probe comprising a T section and an L section. The T section is complementary to a portion of a second domain of the second target mRNA, and the L section is complementary to a nucleic acid component of a signal generating complex.

In some embodiments, first domain of the target mRNA comprises Jβ1 of a TCRβ1 mRNA and the second domain of the target mRNA comprises Cβ1 of a TCRβ1 mRNA, and the first domain of the second target mRNA comprises Jβ2 of a TCRβ2 mRNA and the second domain of the second target mRNA comprises Cβ2 of a TCRβ2 mRNA. In accordance with these embodiments, the method further includes detecting the TCRβ1 mRNA and the TCRβ2 mRNA in the sample using different labels to distinguish TCRβ1 mRNA from TCRβ2 mRNA. In some embodiments, the method further includes evaluating clonality of cells (e.g., T cell clonality) in the sample based on the relative levels (e.g., proportion or ratio) of TCRβ 1 mRNA and TCRβ2 mRNA detected in the sample, which can be indicative of the presence of a disease condition (e.g., a lymphoma), as described further herein. In some embodiments, a predominance of either one of these two signals in a population of cells indicates T cell mono-clonality or clonality, whereas lack of dominance of either signal in a population of cells indicate T cell polyclonality or non-clonality.

Detecting target mRNA(s) using the methods and compositions disclosed herein includes the use of an SGC and/or a label probe comprising a detectable label that corresponds to an mRNA molecule. A label is typically used in RNA in situ hybridization for detecting target nucleic acid. As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In some embodiments, the label is an enzyme. Exemplary enzyme labels include, but are not limited to Horse Radish Peroxidase (HRP), Alkaline Phosphatase (AP), β-galactosidase, glucose oxidase, and the like, as well as various proteases. Other labels include, but are not limited to, fluorophores, Dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the present disclosure, including detectable enzyme/substrate combinations (Pierce, Rockford IL; Santa Cruz Biotechnology, Dallas TX; Life Technologies, Carlsbad CA). In one embodiment of the disclosure, the enzyme can utilize a chromogenic or fluorogenic substrate to produce a detectable signal, as described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. In some embodiments, useful detectable signals are chromogenic or fluorogenic signals. Accordingly, enzymes that are suitable for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactosidase 6-phosphogalactohydrolase, β-glucosidase, αglucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signals are well known to those skilled in the art based on the present disclosure and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN) (4-chloro-1-naphthol), 2,2′-azino-bis (3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-3-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and Quintolet for horseradish peroxidase; 4-Methylumbelliferyl β-D-galactopyranoside, Fluorescein di(β-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in U.S. Patent publication 2012/0100540, which is incorporated herein by reference in its entirety. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

Exemplary rare earth metals and metal isotopes suitable as a detectable label include, but are not limited to, lanthanide (III) isotopes such as ¹⁴¹Pr, ¹⁴²Nd, ¹⁴³Nd, ¹⁴⁴Nd, ¹⁴⁵Nd, ¹⁴⁶Nd, ¹⁴⁷Sm, ¹⁴⁸Nd, ¹⁴⁹Sm, ¹⁵⁰Nd, ¹⁵¹Eu, ¹⁵²Sm, ¹⁵³Eu, ¹⁵⁴Sm, ¹⁵⁵Gd, ¹⁵⁶Gd, ¹⁵⁸Gd, ¹⁵⁹Tb, ¹⁶⁰Gd, ¹⁶¹Dy, ¹⁶²Dy, ¹⁶³Dy, ¹⁶⁴Dy, ¹⁶⁵Ho, ¹⁶⁶Er, ¹⁶⁷Er, ¹⁶⁸Er, ¹⁶⁹Tm, ¹⁷⁰Er, ¹⁷¹Yb, ¹⁷²Yb, ¹⁷³Yb, ¹⁷⁴Yb, ¹⁷⁵Lu, and ¹⁷⁶Yb Metal isotopes can be detected, for example, using time-of-flight mass spectrometry (TOF-MS)(for example, Fluidigm Helios and Hyperion systems, fluidigm.com/systems; South San Francisco, CA).

Biotin-avidin (or biotin-streptavidin) is a well-known signal amplification system based on the fact that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized Horse Radish Peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540, which is incorporated herein by reference in its entirety.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, OR); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway NJ), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NHS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods provided herein can utilize concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the disclosure are well known in the art (see, for example, Johnson and Spence, “Molecular Probes Handbook, a Guide to Fluorescent Probes and Labeling Technologies, 11th ed., Life Technologies (2010)).

Well known methods such as microscopy, cytometry (e.g., mass cytometry, cytometry by time of flight (CyTOF), flow cytometry), or spectroscopy can be utilized to visualize chromogenic, fluorescent, or metal detectable signals associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

As disclosed herein, the label can be designed such that the labels are optionally cleavable. As used herein, a cleavable label refers to a label that is attached or conjugated to a label probe so that the label can be removed, for example, in order to use the same label in a subsequent round of labeling and detecting of target nucleic acids. Generally, the labels are conjugated to the label probe by a chemical linker that is cleavable. Methods of conjugating a label to a label probe so that the label is cleavable are well known to those skilled in the art (see, for example, Hermanson, Bioconjugate Techniques, Academic Press, San Diego (1996); Daniel et al., BioTechniques 24(3):484-489 (1998)). One system of labeling oligonucleotides is the FastTag™ system (Daniel et al., supra, 1998; Vector Laboratories, Burlinghame CA). Various cleavable moieties can be included in the linker so that the label can be cleaved from the label probe. Such cleavable moieties include groups that can be chemically, photo chemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xa (cleavage site IEGR/), tobacco etch virus protease (cleavage site ENLYFQ/G), and thrombin (cleavage site LVPR/GS) (see, for example, Oxford Genetics, Oxford, UK). Another cleavable moiety can be, for example, uracil-DNA (DNA containing uracil), which can be cleaved by uracil-DNA glycosylase (UNG) (see, for example, Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

For methods of the present disclosure for RNA in situ detection of nucleic acid targets in a cell, the cell is optionally fixed and/or permeabilized before hybridization of the target probes. Fixing and permeabilizing cells can facilitate retaining the nucleic acid targets in the cell and permit the target probes, label probes, and so forth, to enter the cell and reach the target nucleic acid molecule. The cell is optionally washed to remove materials not captured to a nucleic acid target. The cell can be washed after any of various steps, for example, after hybridization of the target probes to the nucleic acid targets to remove unbound target probes, and the like. Methods for fixing and permeabilizing cells for in situ detection of nucleic acids, as well as methods for hybridizing, washing and detecting target nucleic acids, are also well known in the art (see, for example, US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss, New York (1995); Ormerod, Flow Cytometry, 2nd ed., Springer (1999)). Exemplary fixing agents include, but are not limited to, aldehydes (formaldehyde, glutaraldehyde, and the like), acetone, alcohols (methanol, ethanol, and the like). Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, CA), and enzymes (for example, lysozyme, lipases, proteases and peptidases). Permeabilization can also occur by mechanical disruption, such as in tissue slices.

RNA in situ detection methods can be used on tissue specimens immobilized on a glass slide, on single cells in suspension such as peripheral blood mononucleated cells (PBMCs) isolated from blood samples, and the like. Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a nucleic acid target that is a biomarker indicative of a pathology.

It is understood by those skilled in the art based on the present disclosure that any of a number of suitable samples can be used for detecting target nucleic acids using methods provided herein. The sample for use in methods provided herein will generally be a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions and/or cells isolated from an organism such as a mammal Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract. Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In some embodiments, the sample is a tissue specimen or is derived from a tissue specimen. In some embodiments, the tissue specimen is formalin-fixed paraffin-embedded (FFPE). In some embodiments, the tissue specimen is fresh frozen. In some embodiments, the tissue specimen is prepared with a fixative other than formalin. In some embodiments, the fixative other than formalin is selected from the group consisting of ethanol, methanol, Bouin's, B5, and I.B.F. In other embodiments, the sample is a blood sample or is derived from a blood sample. In still other embodiments, the sample is a cytological sample or is derived from a cytological sample.

As would be understood by one of ordinary skill in the art based on the present disclosure, embodiments of the compositions and methods provided herein include the ability to measure and/or quantify a detectable label. In some embodiments, the label will be detected using a single-plex format, and in other embodiments, the label will be detected in a duplex or multiplex format, which facilitates the detection and/or quantification of more than one target mRNA. In accordance with these embodiments, the methods and compositions described herein include the use of detection/quantification systems, such as the use of computer software and hardware. In one embodiment, a detection/quantification system of the present disclosure includes a computer and suitable software for receiving user instructions, either in the form of user input into a set of parameter fields (e.g., in a GUI, or in the form of preprogrammed instructions, which can be preprogrammed for a variety of different specific operations to assess a sample). For example, the software can be preprogrammed for one or more operation such as sample handling, slide handling, de-paraffinization, de-crosslinking, hybridization, washing, and the like, as described herein. The software can convert these instructions to appropriate language for controlling the operation of components of the system (e.g., for controlling a fluid handling element and/or laser). The computer can also receive data from other components of the system (e.g., from a detector), and can interpret and/or process the data, provide it to a user in a readable format, or use that data to initiate further operations, in accordance with any programming by the user. In this manner, the system can, for example, quantify any number of detectable labels, compare them to each other (e.g., in a multiplex format) or to controls (e.g., a reference control), and generate a value corresponding to the amount of mRNA in the sample. Any appropriate computer software can be used to facilitate quantitation/detection of a label, including but not limited to, ImageJ, QuPath, and other commercial software such as HALO and VsioPharm.

In some embodiments, the detection/quantification system can measure or quantify target mRNA levels in a sample from a subject suspected of having a disease or condition, and compare those levels to a reference control (e.g., a healthy control sample) in order to determine whether the amount of a given target mRNA(s) with respect to control levels supports a diagnosis that the subject has that disease or condition. In some embodiments, target mRNAs can be compared to each other to determine whether a subject has a disease or condition. In some embodiments, the system can include means for determining proportions or ratios of mRNA target levels with respect to each other and to controls. As would be recognized by one of ordinary skill in the art based on the present disclosure, reference levels or reference controls can be obtained from various sources, including but not limited to, databases of mRNA levels, patient look-up tables, and/or directly from patient samples; which source is used for a given assessment depends on various factors, such as the mRNA target being evaluated, the disease context, the cell/tissue type, and the like.

In some embodiments, the system enables a user (e.g., medical processional such as a pathologist) to view a sample from a subject and compare it to a reference control (from the same subject or a different subject (e.g., healthy control sample). In accordance with these embodiments, the system enables a user to make a determination as to whether a target mRNA is present in sufficient amounts to warrant a diagnosis that the subject has or is likely to develop a disease condition. The system can also provide means for assessing spatial patterns of target mRNA expression and/or determine whether a target mRNA is more or less abundant in a spatially-restricted area of a sample that may correspond to a meaningful anatomical feature (e.g., a tumor micro-environment).

### 4. Therapeutic Methods

Embodiments of the present disclosure also include a method for performing a T cell clonality assay. In accordance with these embodiments, the method includes contacting a sample with a composition to detect the presence of a target mRNA. As described further herein, the composition includes a first target probe pool comprising a plurality of target probes, each target probe comprising a T section and an L section. Each T section of the target probes in the target probe pool is complementary to a portion of a first domain of a target mRNA, and each L section is complementary to a nucleic acid component of an SGC. The composition also includes a separate target probe comprising a T section and an L section, the T section being complementary to a portion of a second domain of the target mRNA and the L section being complementary to a nucleic acid component of an SGC. In some embodiments, the composition also includes a second target probe pool comprising a plurality of target probes, each target probe comprising a T section and an L section, as described above, with each T section complementary to a portion of a first domain of a second target mRNA and each L section complementary to a nucleic acid component of an SGC. The composition further includes a second separate target probe comprising a T section and an L section, the T section being complementary to a portion of a second domain of the second target mRNA, and the L section being complementary to a nucleic acid component of an SGC. In some embodiments, the composition also includes the SGC.

Once the sample is contacted with the composition, the method further includes detecting a signal generated by the SGC corresponding to the target mRNA in the sample, and detecting a different signal generated by the SGC corresponding to the second target mRNA in the sample. In some embodiments, T cell clonality is determined based on the proportion or ratio of each signal generated. In some embodiments, the assay comprises RNA in situ hybridization. In some embodiments, the assay comprises hybridization chain reaction.

In some embodiments, one of the plurality of target probes of the first target probe pool binds the target mRNA, and one of the plurality of target probes of the second target probe pool binds the second target mRNA. In some embodiments, the first domain of the target mRNA comprises Jβ1 of a TCRβ1 mRNA and the second domain of the target mRNA comprises Cβ1 of a TCRβ1 mRNA; and wherein the first domain of the second target mRNA comprises Jβ2 of a TCRβ2 mRNA and the second domain of the second target mRNA comprises Cβ2 of a TCRβ2 mRNA.

In some embodiments, the sample comprises at least one of cell lysate, cell culture, a cell line, a tissue sample, an organ, an organelle, a biological fluid, a mucosa sample, a blood sample, a plasma sample, a urine sample, a skin tissue sample, a vascular tissue sample, a pancreatic tissue sample, a lymphoid tissue sample, a tumor tissue sample, T cell lymphoma tissue, T cells, B cells, and any combination thereof.

In some embodiments, determining T cell clonality based on a proportion of signal(s) generated includes quantifying the signal(s) and comparing to one or more reference levels (e.g., levels corresponding to a non-target mRNA, and/or levels corresponding to a target mRNA comparator (e.g., TCRβ1 or TCRβ2 mRNA levels)). In some embodiments, a predominance of a signal(s) in a population of cells indicates T cell mono-clonality or clonality, whereas lack of dominance of a signal(s) in a population of cells indicates T cell polyclonality or non-clonality. In some embodiments, the sample is a fixed tissue sample, and determining T cell clonality based on the proportion of a signal(s) generated involves assessing spatial distribution of the signal(s) within the tissue sample. In some embodiments, the predominance of either a TCRβ1 or TCRβ2 signal in a region of interest indicates T cell mono-clonality or clonality, whereas a lack of dominance of either signal in the region of interest indicates T cell poly-clonality or non-clonality.

In some embodiments, the method further includes administering a treatment based on determining T cell clonality. In some embodiments, administering a treatment includes CAR T-Cell therapy, chemotherapy, immunotherapy, radiation, drug treatment, stem cell transplantation, surgery, and any combination thereof.

In some embodiments, the compositions and methods provided herein can be used to diagnose and/or treat a disease or condition (e.g., a lymphoma), including evaluating whether a subject with a disease or condition may benefit from a certain type of treatment (e.g., CAR T-cell therapy). As described further herein, embodiments of the present disclosure can include evaluating a subject for the presence/absence of a disease or condition and/or determining treatment options based on T cell clonality. Determining T cell clonality using the methods and compositions provided herein can be used to diagnose and/or treat any disease or condition associated with autoimmune diseases (e.g., lymphomas) as well as immune responses to specific infections (e.g., SARS-CoV-2). In some embodiments, other assays can be performed in conjunction with evaluating a subject for the presence/absence of a disease or condition and/or determining treatment options based on T cell clonality, including but not limited to, RT-PCR, RNAseq, Northern Blot, immunocytochemistry, flow cytometry and the like. In some embodiments, these and other assays can provide validation of an assessment of a subject based on T cell clonality.

In accordance with these embodiments, the composition can include a first target probe pool comprising a plurality of target probes, each target probe comprising a T section and an L section. Each T section of the target probes in the target probe pool is complementary to a portion of a first domain of a target mRNA, and each L section is complementary to a nucleic acid component of an SGC. The composition also includes a separate target probe comprising a T section and an L section, the T section being complementary to a portion of a second domain of the target mRNA and the L section being complementary to a nucleic acid component of an SGC. In some embodiments, the composition also includes a second target probe pool comprising a plurality of target probes, each target probe comprising a T section and an L section, as described above, with each T section complementary to a portion of a first domain of a second target mRNA and each L section complementary to a nucleic acid component of an SGC. The composition further includes a second separate target probe comprising a T section and an L section, the T section being complementary to a portion of a second domain of the second target mRNA, and the L section being complementary to a nucleic acid component of an SGC. In some embodiments, the composition also includes the SGC. In some embodiments, a first signal is generated corresponding to an amount of the target mRNA in the sample and wherein a second signal is generated corresponding to an amount of the second target mRNA in the sample. In some embodiments, the subject is diagnosed as having a lymphoma based on a comparison of the signals generated.

In some embodiments, the compositions, kits, and methods of the present disclosure can include labels or inserts, such as instructions for performing an assay. Labels or inserts include information on a condition, disorder, disease, or symptom for which the kit component may be used. Labels or inserts can include instructions for a clinician or for a subject to use one or more of the kit components in a method, treatment protocol, or therapeutic regimen. In some embodiments, labels or inserts include information on cancers for which the kit component may be used for, such as colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT), breast secretory carcinoma, infantile (congenital) fibrosarcoma, mammary analogue secretory carcinoma of salivary glands, congenital mesoblastic nephroma, spitz tumors, intrahepatic cholangiocarcinoma, appendiceal adenocarcinoma, pediatric DIPG and non-brainstem high-grade glioma, uterine sarcoma, thyroid carcinoma, sarcoma (NOS), GIST, lung adenocarcinoma, ph-like acute lymphoblastic leukemia, colon adenocarcinoma, brain low-grade glioma, or breast invasive carcinoma. In some embodiments, labels or inserts include information on cancers for which the kit component may be used for, such as mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, labels or inserts include information on cancers for which the kit component may be used for a hematological cancer, such as Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia.

In some embodiments, the labels or inserts include instructions for a clinician, pathologist, or other medical professional to interpret data produced by the reagents from the kits and compositions described herein on a subject, including whether and when to employ other detection technology, and subsequently to make a decision on whether to administer a treatment to the subject. In some embodiments, if an RNA in situ hybridization assay performed using the compositions of the present disclosure produces positive staining from a sample subject (e.g., indicative of T cell clonality), a clinician can begin administering a treatment for the subject.

In some embodiments, the method includes administering a treatment based on determining T cell clonality, which can be indicative of the presence of a lymphoma in a subject (e.g., T cell lymphoma such as non-Hodgkin lymphoma). In some embodiments, administering a treatment includes CAR T-Cell therapy, chemotherapy, immunotherapy, radiation, drug treatment, stem cell transplantation, surgery, and any combination thereof. For example, upon a positive test indicating T cell clonality using the compositions and methods described herein, a subject can be administered a treatment according to a certain treatment regimen for a lymphoma. In some embodiments, the treatment can include administering a pharmaceutical composition that includes one or more of doxorubicin (Adriamycin), bleomycin (Blenoxane), vinblastine (Velban), dacarbazine (DTIC-Dome), etoposide (Etopophos, Toposar, VePesid, VP-16), cyclophosphamide (Cytoxan), vincristine (Oncovin), procarbazine (Matulane), prednisone (prednisone (Rayos, Prednisone Intensol), chlorambucil (Leukeran), bendamustine (Treanda), ifosfamide (Ifex), carboplatin (Paraplatin), cisplatin (Platinol), oxaliplatin (Eloxatin), cladribine (2-CdA, Leustatin), fludarabine (Fludera), pentostatin (Nipent), capecitabine (Xeloda), cytarabine (ara-C), gemcitabine (Gemzar), methotrexate (Trexall), pralatrexate (Folotyn), brentuximab vedotin (Adcetris), pralatrexate (Folotyn), bortezomib (Velcade), belinostat (Beleodaq), or romidepsin (Istodax), alemtuzumab (Campath) and denileukin diftitox (Ontak), and any combination thereof. In some embodiments, additional therapy may include antibody-drug conjugates targeting CD37, anti-CD47-based therapies, bispecific antibodies targeting both CD30 and CD16A, and other currently available biologics. These compounds and biologics can be formulated into suitable pharmaceutical compositions for different routes of administration, such as injection, sublingual and buccal, rectal, vaginal, ocular, otic, nasal, inhalation, nebulization, cutaneous, or transdermal, for example, using techniques and procedures well known in the art (see, e.g., Ansel, Introduction to Pharmaceutical Dosage Forms, (7th ed. 1999)).

In some embodiments, the method includes administering a treatment to target T cell receptor β, including, but not limited to, CAR-T cell immunotherapy (e.g., see Maciocia, P. M. et al. (2017), Targeting the T cell receptor β-chain constant region for immunotherapy of T cell malignancies. Nature Medicine, 23(12), 1416-1423)).

The subject or patient administered a therapy provided herein can be a mammal. In one embodiment, the subject is a human. In another embodiment, the subject is a human with cancer. In some embodiments, the cancer is selected from a group consisting of mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the cancer is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

Various delivery systems are known and can be used to administer a pharmaceutical composition provided herein, including, but not limited to, parenteral administration (e.g., intradermal, intramuscular, intraperitoneal, intravenous and subcutaneous), epidural, and mucosal (e.g., intranasal and oral routes). In one embodiment, a pharmaceutical composition is administered intranasally, intramuscularly, intravenously, or subcutaneously. In another embodiment, a pharmaceutical composition is administered orally. The pharmaceutical composition may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent.

The amount of a pharmaceutical composition provided herein that will be effective in the prevention and/or treatment of cancer can be determined by standard clinical techniques. In addition, in vitro assays may optionally be employed to help identify optimal dosage ranges. The precise dose to be employed in the formulation will also depend on the route of administration, and the seriousness of a disease or condition, and in some embodiments, should be decided according to the judgment of the practitioner and each patient's circumstances. Effective doses may also be extrapolated from dose-response curves derived from in vitro or animal model test systems.

The dose administered to a mammal, particularly a human, in the context of the present disclosure should be sufficient to affect a therapeutic response in the mammal over a reasonable time frame. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration.

The pharmaceutical composition can be delivered as a single dose (e.g., a single bolus injection), or over time (e.g., continuous infusion over time or divided bolus doses over time). The agent can be administered repeatedly if necessary, for example, until the patient experiences stable disease or regression, or until the patient experiences disease progression or unacceptable toxicity. Stable disease or lack is determined by methods known in the art such as evaluation of patient symptoms, physical examination, and visualization of the tumor that has been imaged using X-ray, CAT, PET, MRI scan, or other commonly accepted evaluation modalities.

The pharmaceutical composition can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In certain embodiments, the compound is administered once per day from one day to six months, from one week to three months, from one week to four weeks, from one week to three weeks, or from one week to two weeks.

### 5. Kits

Embodiments of the present disclosure also include a kit for detecting an mRNA target. In accordance with these embodiments, the kit includes a first target probe pool comprising a plurality of target probes. As described further herein, each target probe comprising a T section and an L section, and each T section is complementary to a portion of a first domain of a target mRNA and each L section is complementary to a nucleic acid component of an SGC. The kit can also include a first target probe comprising a T section and an L section, the T section being complementary to a portion of a second domain of the target mRNA and the L section being complementary to a nucleic acid component of an SGC. In some embodiments, the kit also includes an SGC and instructions for performing a hybridization reaction to detect the mRNA target.

In some embodiments, the SGC includes a label probe, and optionally, one or more of an amplifier, a pre-amplifier, and a pre-pre-amplifier. In some embodiments, the label probe includes at least one detectable label. In one embodiment, the kit provided herein comprises agents for performing RNAscope® as described in more detail in, e.g., U.S. Pat. Nos. 7,709,198, 8,604,182, and 8,951,726, which are herein incorporated by reference in their entireties. In another embodiment, the kit provided herein comprises agents for performing BaseScope™, which is described in more detail in, e.g., U.S. patent application Ser. No. 13/575,936, and PCT Appln. No. PCT/US2011/023126, which are incorporated herein by reference in their entireties. In some embodiments, the kit comprises at least one set of two or more target probes capable of hybridizing to a target nucleic acid, and an SGC capable of hybridizing to the set of two or more target probes. In some embodiments, the SGC comprises a label probe and a nucleic acid component capable of hybridizing to the set of two or more target probes, the target nucleic acid comprising a region of mRNA encoding a kinase domain of T cell receptor.

In some embodiments, the kit further includes other agents or materials for performing RNA in situ hybridization or hybridization chain reaction assays, including but not limited to, fixing agents and agents for treating samples for preparing hybridization, agents for washing samples, and the like. In some embodiments, the kit includes at least one of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

In some embodiments, the kit further includes a calibrator or control polynucleotide. In some embodiments, the calibrator or control polynucleotide comprises a sequence complementary to a portion of any one of SEQ ID NOs: 1-14. In some embodiments, the calibrator or control polynucleotide comprises a sequence identical to a portion of any one of SEQ ID NOs: 1-14.

In some embodiments, the kit further includes a second target probe pool comprising a plurality of target probes. Each target probe comprises a T section and an L section, and each T section is complementary to a portion of a first domain of a second target mRNA and each L section is complementary to a nucleic acid component of an SGC. The kit can further include a second target probe comprising a T section that is complementary to a portion of a second domain of the second target mRNA, and an L section is complementary to a nucleic acid component of a signal generating complex.

The kits of the present disclosure may further include instructions and/or packaging material, which generally includes to a physical container for housing and/or delivering the components of the kit. The packaging material can maintain the components sterilely, and can be made of material commonly used for such purposes (e.g., paper, corrugated fiber, glass, plastic, foil, ampules, vials, tubes, etc.).

Kits provided herein can include labels or inserts, such as instructions for performing an assay. Labels or inserts include information on a condition, disorder, disease, or symptom for which the kit component may be used. Labels or inserts can include instructions for a clinician or for a subject to use one or more of the kit components in a method, treatment protocol, or therapeutic regimen. In some embodiments, labels or inserts include information on cancers for which the kit component may be used for, such as colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT), breast secretory carcinoma, infantile (congenital) fibrosarcoma, mammary analogue secretory carcinoma of salivary glands, congenital mesoblastic nephroma, spitz tumors, intrahepatic cholangiocarcinoma, appendiceal adenocarcinoma, pediatric DIPG and non-brainstem high-grade glioma, uterine sarcoma, thyroid carcinoma, sarcoma (NOS), GIST, lung adenocarcinoma, ph-like acute lymphoblastic leukemia, colon adenocarcinoma, brain low-grade glioma, or breast invasive carcinoma. In some embodiments, labels or inserts include information on cancers for which the kit component may be used for, such as mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, labels or inserts include information on cancers for which the kit component may be used for a hematological cancer, such as Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In some embodiments, the labels or inserts include instructions for a clinician, pathologist, or other medical professional to interpret data produced by the reagents from the kits and compositions described herein on a subject, including whether and when to employ other detection technology, and subsequently to make a decision on whether to administer a treatment to the subject.

Labels or inserts can include “printed matter,” e.g., paper or cardboard, separate or affixed to a component, a kit or packing material (e.g., a box), or attached to, for example, an ampule, tube or vial containing a kit component. Labels or inserts can additionally include a computer readable medium, such as a disk (e.g., hard disk, card, and memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date. In some embodiments, the kit provided herein is for determination if a subject has cancer or is likely to develop cancer (e.g., a lymphoma).

### 6. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

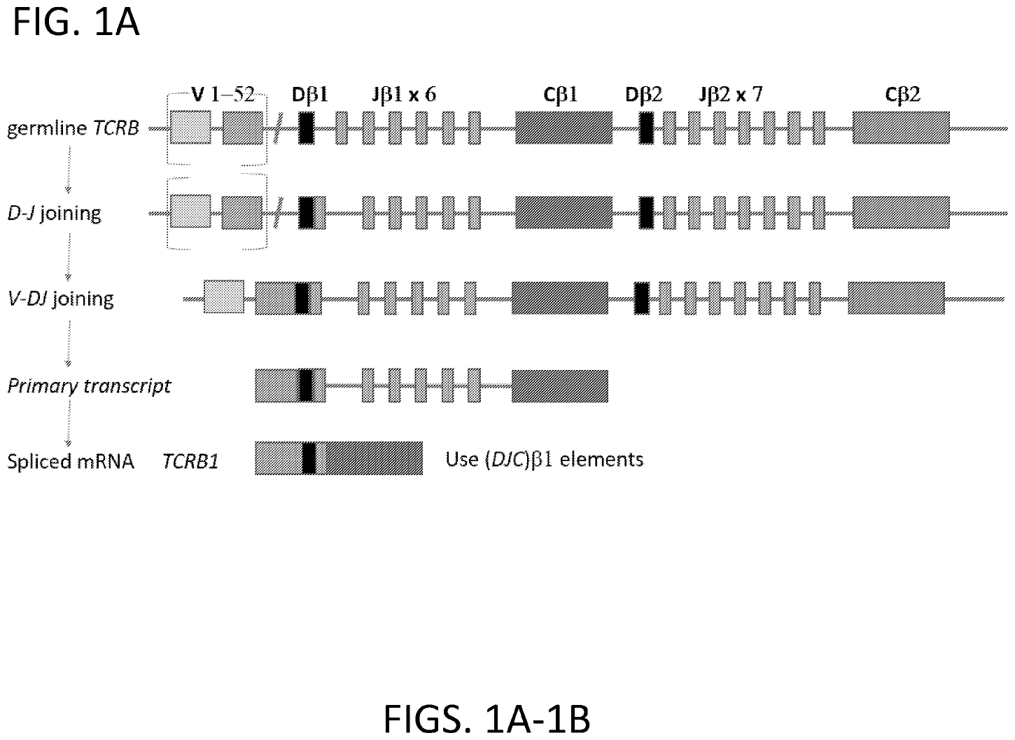

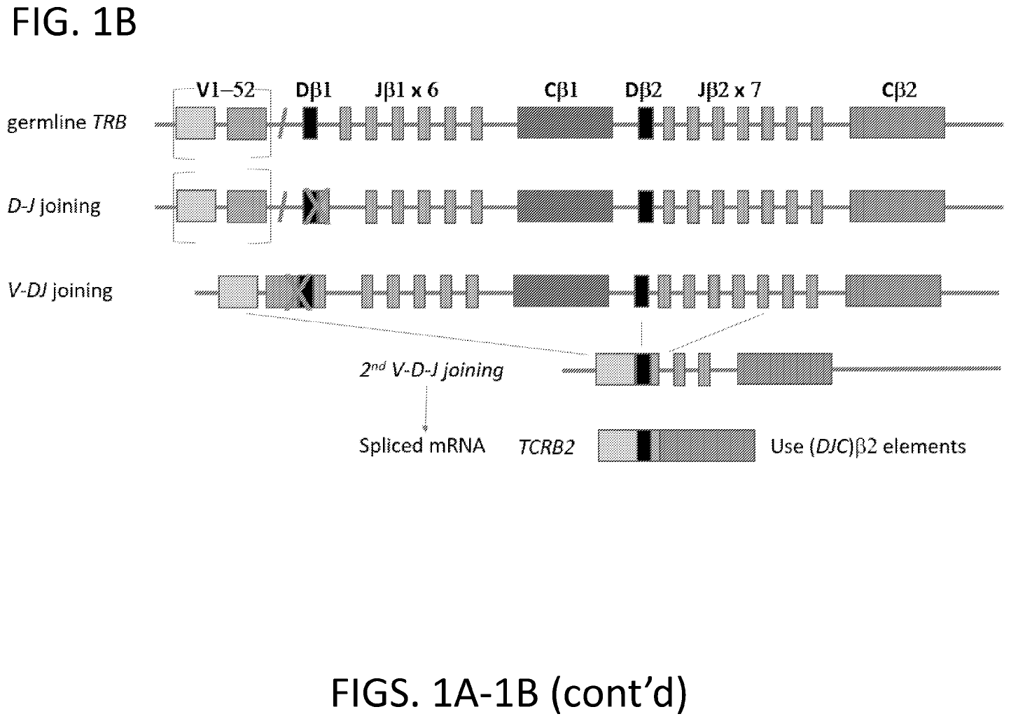

In one embodiment, the compositions and methods provided herein can be used to provide a novel method for T cell clonality testing. The TCR β locus is located in chromosome band 7q35 and is rearranged in the vast majority (>95%) of T cell malignancies to express the β chain of TCR. The TCRB locus consists of 52 V regions, 2 D regions, 2 J clusters and 2 C regions (FIG. 1A). The C gene segments Cβ1 and Cβ2 are preceded by 6 Jβ1 segments (Jβ1.1-Jβ1.6) and 7 Jβ2 segments (Jβ2.1-Jβ2.7), respectively. The TCRB rearrangement starts with the joining of Dβ1 to one of Jβ1 elements, followed by joining one of the V segments with DJ. Transcription of the rearranged locus and splicing of the primary transcript generate the TCRB1 transcript that joins VDJ and the Cβ1 region. However, either D-J or V-DJ joining events can be nonproductive due to the out-of-frame joining of the two sequences. When this occurs (1/9 chance), the rescue pathway rearranges the V-(D-J)β2 segments to generate the TCRB2 mRNA (FIG. 1B). As a result, a T cell either expresses TCRB1 or TCRB2 mRNA. Therefore, T cell clonality can be determined by detecting and distinguishing between TCRB1 and TCRB2 mRNA transcript variants.

The Cβ1 and Cβ2 sequences are the most readily apparent targets for detecting TCRB1 and TCRB2 mRNA, respectively, as they are not differentially spliced when pre-mRNA is converted into mature mRNA. However, Cβ1 and Cβ2 sequences are 99% identical, making them difficult to distinguish. Alternatively, the sequences of the Jβ1 domains are much more divergent from those of Jβ2 domains (<70% identity); however, they are differentially spliced during pre-mRNA processing, making it difficult to determine which of the Jβ1 and Jβ2 domains will ultimately be present in the fully spliced mature mRNA, which complicates target mRNA detection.

Although determining which Jβ1 and Jβ2 elements will ultimately be incorporated in a given TCRB1 or TCRB2 mRNA transcript variant is unpredictable, the methods and compositions of the present disclosure address this challenge using one or more oligo probes specific to each of the Jβ1 and Jβ2 elements (FIG. 2). All Jβ1 oligos (Z₁) are pooled into a single probe pool to detect any Jβ1 element, and all Jβ2 oligos (Z₁) are pooled into a single probe pool to detect any Jβ2 element. The Jβ1 oligo pool and Jβ2 oligo pool can then be used to specifically detect TCRB1 and TCRB2 mRNA, respectively. The specificity of TCRB1 and TCRB2 mRNA detection can be further enhanced by using a paired probe strategy. This paired probe strategy uses a second oligo targeting the beginning Cβ1 and Cβ2 sequences. With this strategy, only when one of the Jβ probes (Z₁) and the Cβ probe (Z₂) both bind to the fully spliced TCRB1 or TCRB2 mRNA is a detectable signal generated, which further enhances detection specificity. Example designs of oligo probes for Jβ1 and Jβ2 are shown in the sequence listing provided below. Also provided in the sequence listing are example designs of oligo probes for Cβ1 and Cβ2, which are used with the Jβ1 and Jβ2 oligos for paired probe signal amplification and detection technologies. These probes for (J-C)β1 and (J-C)β2 can be used either in two single-plex assays (e.g., detecting TCRB1 and TCRB2 separately in two slides), or in a duplex assay (e.g., detecting both TCRB1 and TCRB2 simultaneously using differently colored labels).

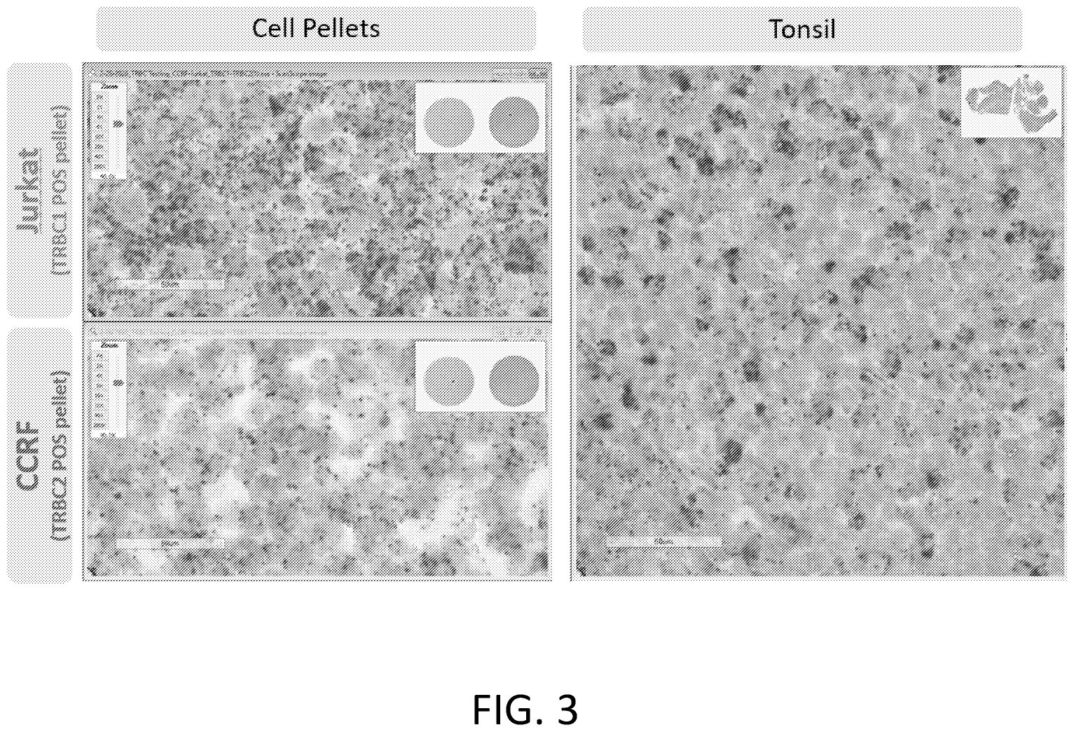

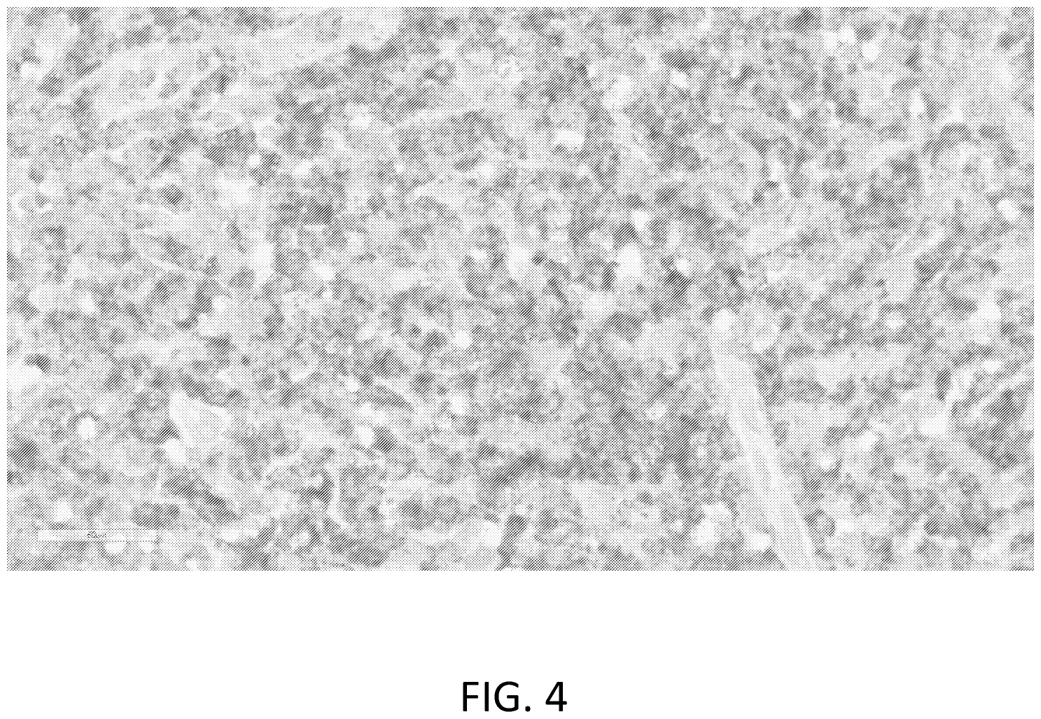

This probe design strategy used a variant of a BaseScope™ assay to detect two well characterized T-cell acute lymphoblastic leukemia derived cell lines, Jurkat and CCRF. As shown in FIG. 3, the duplex assay demonstrated exclusive expression of TCRB1 in Jurkat and TCRB2 in CCRF cells, consistent with the known usage of Jβ1.2 in Jurkat and Jβ2.3 in CCRF, respectively. In contrast, in the human Tonsil tissue, the duplex assay detected a heterogeneous population of cells expressing either TCRB1 or TCRB2, demonstrating the expected polyclonal staining pattern. Thus, these data demonstrated the ability of the compositions and methods of the present disclosure to detect monoclonal and polyclonal T cell populations in a patient sample. As a further example in a clinically relevant tissue, a T cell lymphoma tumor tissue section demonstrated monoclonal expression of TCRB1 (FIG. 4), consistent with the monoclonal origin of this tumor.

The RISH-based methods and compositions provided herein avoids the many challenges and limitations associated with conventional PCR-based methods. First, this RISH-based method detects RNA targets directly on a slide, thus preserving the morphological/histological information lost in PCR-based assays. This allows a pathologist to view the slides in the context of tissue architecture and cellular morphology and interpret the results with greater accuracy. Second, PCR-based methods may not amplify all targeted regions equally efficiently. This amplification bias can be caused by different amplicon sizes and the suboptimal DNA quality present in archival FFPE tissues. Third, PCR-based methods require relatively large amounts of DNA inputs for multiple PCR reactions, whereas the slide-based RISH assay requires only one or two 5-micron tissue sections, making it much more robust when sample material is limited and heterogeneous. Fourth, the RISH-based assay generates clear staining patterns that can be directly viewed and scored under a standard microscope (e.g., chromogenic reactions), avoiding the limitations of pseudo-clonality due to limited DNA input during PCR and the need to interpret complex fragment patterns. Finally, the RISH assays described herein can be fully automated with a rapid turnaround time (e.g., less than one day), compared to the complicated and time-consuming protocols of PCR-based clonality testing. This method is broadly accessible in a clinical setting and useful in both diagnostic and minimum residual disease monitoring applications.

### 7. Sequences

Sequences relevant to the embodiments of the present disclosure are provided in the table below. TRB Jβ1 and Jβ2 sequences comprise regions complementary to corresponding oligo probes (e.g., schematically shown as Z₁ in FIG. 2). TRB Cβ1 and Cβ2 sequences comprise regions complementary to corresponding oligo probes (e.g., schematically shown as Z₂ in FIG. 2).

TABLE 1
Sequence information.

| | | Accession | SEQ ID |
|---|---|---|---|
| Name | Sequence | No. | NO: |
| Jβ1-1 | tgaacactgaagctttcttt | K02545 | 1 |
| | ggacaaggcaccagactcac | | |
| | agttgtag | | |
| Jβ1-2 | ctaactatggctacaccttc | K02545 | 2 |
| | ggttcggggaccaggttaac | | |
| | cgttgtag | | |
| Jβ1-3 | ctctggaaacaccatatatt | M14158 | 3 |
| | ttggagagggaagttggctc | | |
| | actgttgtag | | |
| Jβ1-4 | caactaatgaaaaactgttt | M14158 | 4 |
| | tttggcagtggaacccagct | | |
| | ctctgtcttgg | | |
| Jβ1-5 | tagcaatcagccccagcatt | M14158 | 5 |
| | ttggtgatgggactcgactc | | |
| | tccatcctag | | |
| Jβ1-6 | ctcctataattcacccctcc | M14158 | 6 |
| | actttgggaatgggaccagg | | |
| | ctcactgtgacag | | |
| Jβ2-1 | ctcctacaatgagcagttct | X02987 | 7 |
| | tcgggccagggacacggctc | | |
| | accgtgctag | | |
| Jβ2-2 | cgaacaccggggagctgttt | X02987 | 8 |
| | tttggagaaggctctaggct | | |
| | gaccgtactgg | | |
| Jβ2-2P | ctgagaggogctgctgggcg | X02987 | 9 |
| | tctgggcggaggactcctgg | | |
| | ttctgg | | |
| Jβ2-3 | agcacagatacgcagtattt | X02987 | 10 |
| | tggcccaggcacccggctga | | |
| | cagtgctcg | | |
| Jβ2-4 | agccaaaaacattcagtact | X02987 | 11 |
| | tcggcgccgggacccggctc | | |
| | tcagtgctgg | | |
| Jβ2-5 | accaagagacccagtacttc | X02987 | 12 |
| | gggccaggcacgcggctcct | | |
| | ggtgctcg | | |
| Jβ2-6 | ctctggggccaacgtcctga | X02987 | 13 |
| | ctttcggggccggcagcagg | | |
| | ctgaccgtgctgg | | |
| Jβ2-7 | ctcctacgagcagtacttcg | M14159 | 14 |
| | ggccgggcaccaggctcacg | | |
| | gtcacag | | |
| Cβ1 | gaggacctgaacaaggtgtt | M12887 | 15 |
| | cccacccgaggtcgctgtgt | | |
| | ttgagccatcagaagcagag | | |
| | atctcccacacccaaaaggc | | |
| | cacactggtgtgcctggcca | | |
| | caggcttcttccccgaccac | | |
| | gtggagctgagctggtgggt | | |
| | gaatgggaaggaggtgcaca | | |
| | gtggggtcagcacggacccg | | |
| | cagcccctcaaggagcagcc | | |
| | cgccctcaatgactccagat | | |
| | actgcctgagcagccgcctg | | |
| | agggtctcggccaccttctg | | |
| | gcagaacccccgcaaccact | | |
| | tccgctgtcaagtccagttc | | |
| | tacgggctctcggagaatga | | |
| | cgagtggacccaggataggg | | |
| | ccaaacccgtcacccagate | | |
| | gtcagcgccgaggcctgggg | | |
| | tagagca | | |
| Cβ2 | gaggacctgaaaaacgtgtt | M12888 | 16 |
| | cccacccgaggtcgctgtgt | | |
| | ttgagccatcagaagcagag | | |
| | atctcccacacccaaaaggc | | |
| | cacactggtgtgcctggcca | | |
| | caggcttctaccccgaccac | | |
| | gtggagctgagctggtgggt | | |
| | gaatgggaaggaggtgcaca | | |
| | gtggggtcagcacagacccg | | |
| | cagcccctcaaggagcagcc | | |
| | cgccctcaatgactccagat | | |
| | actgcctgagcagccgcctg | | |
| | agggtctcggccaccttctg | | |
| | gcagaacccccgcaaccact | | |
| | tccgctgtcaagtccagttc | | |
| | tacgggctctcggagaatga | | |
| | cgagtggacccaggataggg | | |
| | ccaaacctgtcacccagatc | | |
| | gtcagegccgaggcctgggg | | |
| | tagagca | | |

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

What is claimed is:

1. A composition comprising:
a first target probe pool comprising a plurality of target probes, each target probe comprising a T section and an L section, wherein each T section is complementary to a portion of a first domain of a target mRNA and wherein each L section is complementary to a nucleic acid component of a signal generating complex;
a first target probe comprising a T section and an L section, wherein the T section is complementary to a portion of a second domain of the target mRNA and wherein the L section is complementary to a nucleic acid component of a signal generating complex; and
a signal generating complex.

2. The composition of claim 1, wherein each of the target probes of the first target probe pool comprises a T section that is complementary to a non-overlapping portion of the first domain of the target mRNA.

3. The composition of claim 1 or claim 2, wherein the T section of the first target probe is complementary to a portion of a second domain of the target mRNA that is adjacent to the first domain of the target mRNA.

4. The composition of any of claims 1 to 3, wherein the T section of at least one of the target probes of the first target probe pool is 3′ of its L section.

5. The composition of any of claims 1 to 3, wherein the T section of at least one of the target probes of the first target probe pool is 5′ of its L section.

6. The composition of any of claims 1 to 5, wherein the T section of the first target probe is 3′ of its L section.

7. The composition of any of claims 1 to 5, wherein the T section of the first target probe is 5′ of its L section.

8. The composition of any of claims 1 to 7, wherein at least one of the target probes of the first target probe pool and/or the first target probe form a hairpin structure.

9. The composition of any of claims 1 to 8, wherein the signal generating complex comprises a label probe, and optionally, one or more of an amplifier, a pre-amplifier, and a pre-pre-amplifier.

10. The composition of claim 9, wherein the label probe comprises the nucleic acid component of the signal generating complex which binds the L section of at least one of the target probes of the first target probe pool and the L section of the first target probe.

11. The composition of claim 10, wherein the label probe comprises at least one detectable label.

12. The composition of any of claims 1 to 11, wherein the target mRNA comprises a portion of a T cell receptor (TCR).

13. The composition of any of claims 1 to 12, wherein the first domain of the target mRNA comprises Jβ1 or Jβ2 of a TCRβ1 or a TCRβ2 mRNA, respectively.

14. The composition of any of claims 1 to 13, wherein the T sections of the plurality of target probes of the first target probe pool are complementary to at least a portion of any of SEQ ID NOs: 1-6.

15. The composition of any of claims 1 to 13, wherein the T sections of the plurality of target probes of the first target pool are complementary to at least a portion of any of SEQ ID NOs: 7-14.

16. The composition of any of claims 1 to 15, wherein the second domain of the target mRNA comprises Cβ1 or Cβ2 of a TCRβ1 or a TCRβ2 mRNA, respectively.

17. The composition of any of claims 1 to 16, wherein the T section of the first target probe is complementary to at least a portion of either of SEQ ID NOs: 15 or 16.

18. The composition of any of claims 1 to 17, wherein the composition further comprises a second target probe pool comprising a plurality of target probes, each target probe comprising a T section and an L section, wherein each T section is complementary to a portion of a first domain of a second target mRNA and wherein each L section is complementary to a nucleic acid component of a signal generating complex.

19. The composition of any of claims 1 to 18, wherein the target mRNA complementary to the plurality of target probes in the first target probe pool is TCRβ1, and wherein the second target mRNA complementary to the plurality of target probes in the second target probe pool is TCRβ2.

20. The composition of any of claims 1 to 19, wherein the composition further comprises a second target probe comprising a T section and an L section, wherein the T section is complementary to a portion of a second domain of the second target mRNA, and wherein the L section is complementary to a nucleic acid component of a signal generating complex.

21. The composition of any of claims 1 to 20, wherein the target mRNA complementary to the first target probe is TCRβ1, and wherein the second target mRNA complementary to the second target probe is TCRβ2.

22. The composition of any of claims 1 to 21, wherein the composition further comprises a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

23. A method of detecting an mRNA target, the method comprising:
(i) contacting a sample with a composition comprising:
a first target probe pool comprising a plurality of target probes, each target probe comprising a T section and an L section, wherein each T section is complementary to a portion of a first domain of a target mRNA and wherein each L section is complementary to a nucleic acid component of a signal generating complex;
a first target probe comprising a T section and an L section, wherein the T section is complementary to a portion of a second domain of the target mRNA and wherein the L section is complementary to a nucleic acid component of a signal generating complex; and
a signal generating complex; and
(ii) detecting a signal generated by the signal generating complex corresponding to the target mRNA in the sample.

24. The method of claim 23, wherein the method comprises RNA in situ hybridization.

25. The method of claim 23, wherein the method comprises hybridization chain reaction.

26. The method of any of claims 23 to 25, wherein one of the plurality of target probes of the first target probe pool binds the target mRNA.

27. The method of any of claims 23 to 26, wherein the nucleic acid portion of the signal generating complex binds both the L section of one of the plurality of target probes of the first target probe pool and the L section of the first target probe.

28. The method of any of claims 23 to 27, wherein the composition further comprises:
a second target probe pool comprising a plurality of target probes, each target probe comprising a T section and an L section, wherein each T section is complementary to a portion of a first domain of a second target mRNA and wherein each L section is complementary to a nucleic acid component of a signal generating complex; and
a second target probe comprising a T section and an L section, wherein the T section is complementary to a portion of a second domain of the second target mRNA, and wherein the L section is complementary to a nucleic acid component of a signal generating complex.

29. The method of any of claims 23 to 28, wherein the first domain of the target mRNA comprises Jβ1 of a TCRβ 1 mRNA and the second domain of the target mRNA comprises Cβ1 of a TCRβ 1 mRNA; and wherein the first domain of the second target mRNA comprises Jβ2 of a TCRβ2 mRNA and the second domain of the second target mRNA comprises Cβ2 of a TCRβ2 mRNA.

30. The method of claim 29, wherein the method further comprises detecting the TCRβ1 mRNA and the TCRβ2 mRNA in the sample simultaneously using different labels.

31. The method of claim 29, wherein the method further comprises detecting the TCRβ1 mRNA and the TCRβ2 mRNA in the sample separately using the same or different labels.

32. The method of claim 30 or claim 31, further comprising evaluating clonality of cells in the sample based on the proportion of TCRβ1 mRNA and TCRβ2 mRNA detected in the sample.

33. A kit for detecting an mRNA target, the kit comprising:
a first target probe pool comprising a plurality of target probes, each target probe comprising a T section and an L section, wherein each T section is complementary to a portion of a first domain of a target mRNA and wherein each L section is complementary to a nucleic acid component of a signal generating complex;
a first target probe comprising a T section and an L section, wherein the T section is complementary to a portion of a second domain of the target mRNA and wherein the L section is complementary to a nucleic acid component of a signal generating complex;
a signal generating complex; and
instructions for performing a hybridization reaction to detect the mRNA target.

34. The kit of claim 33, wherein the signal generating complex comprises a label probe, and optionally, one or more of an amplifier, a pre-amplifier, and a pre-pre-amplifier.

35. The kit of claim 33 or claim 34, wherein the label probe comprises at least one detectable label.

36. The kit of any of claims 33 to 35, wherein the kit further comprises at least one of a hybridization buffer, dextran sulfate, formamide, dithiothreitol (DDT), sodium chloride and sodium citrate (SSC), EDTA, Denhardt's solution, a fluorescent label, a chromogenic label, dNTPs, single-stranded DNA, tRNA, polyA, an initiator oligo, or any combination thereof.

37. The kit of any of claims 33 to 36, wherein the kit further comprises a calibrator or control polynucleotide.

38. The kit of claim 37, wherein the calibrator or control polynucleotide comprises a sequence complementary to a portion of any one of SEQ ID NOs: 1-16.

39. The kit of claim 37, wherein the calibrator or control polynucleotide comprises a sequence identical to a portion of any one of SEQ ID NOs: 1-16.

40. The kit of any of claims 33 to 39, wherein the kit further comprises:
a second target probe pool comprising a plurality of target probes, each target probe comprising a T section and an L section, wherein each T section is complementary to a portion of a first domain of a second target mRNA and wherein each L section is complementary to a nucleic acid component of a signal generating complex; and
a second target probe comprising a T section and an L section, wherein the T section is complementary to a portion of a second domain of the second target mRNA, and wherein the L section is complementary to a nucleic acid component of a signal generating complex.

41. A method for performing a T cell clonality assay, the method comprising:
(i) contacting a sample with a composition comprising:
a first target probe pool comprising a plurality of target probes, each target probe comprising a T section and an L section, wherein each T section is complementary to a portion of a first domain of a target mRNA and wherein each L section is complementary to a nucleic acid component of a signal generating complex;
a first target probe comprising a T section and an L section, wherein the T section is complementary to a portion of a second domain of the target mRNA and wherein the L section is complementary to a nucleic acid component of a signal generating complex;
a second target probe pool comprising a plurality of target probes, each target probe comprising a T section and an L section, wherein each T section is complementary to a portion of a first domain of a second target mRNA and wherein each L section is complementary to a nucleic acid component of a signal generating complex;
a second target probe comprising a T section and an L section, wherein the T section is complementary to a portion of a second domain of the second target mRNA, and wherein the L section is complementary to a nucleic acid component of a signal generating complex; and
a signal generating complex; and
(ii) detecting a signal generated by the signal generating complex corresponding to the target mRNA in the sample, and detecting a different signal generated by the signal generating complex corresponding to the second target mRNA in the sample;
wherein T cell clonality is determined based on the proportion of each signal generated.

42. The method of claim 42, wherein the assay comprises RNA in situ hybridization.

43. The method of claim 42, wherein the assay comprises hybridization chain reaction.

44. The method of claim 42 or claim 43, wherein one of the plurality of target probes of the first target probe pool binds the target mRNA, and one of the plurality of target probes of the second target probe pool binds the second target mRNA.

45. The method of any of claims 42 to 44, wherein the first domain of the target mRNA comprises Jβ1 of a TCRβ 1 mRNA and the second domain of the target mRNA comprises Cβ1 of a TCRβ 1 mRNA; and wherein the first domain of the second target mRNA comprises Jβ2 of a TCRβ2 mRNA and the second domain of the second target mRNA comprises Cβ2 of a TCRβ2 mRNA.

46. The method of any of claims 42 to 45, wherein the sample comprises at least one of cell lysate, cell culture, a cell line, a tissue sample, an organ, an organelle, a biological fluid, a mucosa sample, a blood sample, a plasma sample, a urine sample, a skin tissue sample, a vascular tissue sample, a pancreatic tissue sample, a lymphoid tissue sample, a tumor tissue sample, T cell lymphoma tissue, T cells, B cells, and any combination thereof.

47. The method of any of claims 42 to 46, wherein determining T cell clonality based on the proportion of each signal generated comprises quantifying the signals and comparing relative levels of signals of TCRβ1 and TCRβ2 mRNAs.

48. The method of any of claims 42 to 47, wherein the sample is a fixed tissue sample, and wherein determining T cell clonality based on the proportion of each signal generated comprises assessing spatial distribution of the signals within the tissue sample.

49. The method of any of claims 42 to 48, wherein the method further comprises administering a treatment based on determining T cell clonality.

50. The method of claim 49, wherein administering a treatment comprises CAR T-Cell therapy, chemotherapy, immunotherapy, radiation, drug treatment, stem cell transplantation, surgery, and any combination thereof.

51. A composition for use in diagnosing lymphoma, the composition comprising:
a first target probe pool comprising a plurality of target probes, each target probe comprising a T section and an L section, wherein each T section is complementary to a portion of a first domain of a target mRNA and wherein each L section is complementary to a nucleic acid component of a signal generating complex;
a first target probe comprising a T section and an L section, wherein the T section is complementary to a portion of a second domain of the target mRNA and wherein the L section is complementary to a nucleic acid component of a signal generating complex;
a second target probe pool comprising a plurality of target probes, each target probe comprising a T section and an L section, wherein each T section is complementary to a portion of a first domain of a second target mRNA and wherein each L section is complementary to a nucleic acid component of a signal generating complex;
a second target probe comprising a T section and an L section, wherein the T section is complementary to a portion of a second domain of the second target mRNA, and wherein the L section is complementary to a nucleic acid component of a signal generating complex; and
a signal generating complex;
wherein a first signal is generated corresponding to an amount of the target mRNA in the sample and wherein a second signal is generated corresponding to an amount of the second target mRNA in the sample, and wherein the subject is diagnosed as having a lymphoma based on a comparison of the signals generated.
